SEQUENCE LISTING (8 sequences)

SEQ ID NO: 1 - DNA, 20 bp, Artificial Sequence
  Synthesized
ttgtgtccaccaggatctca

SEQ ID NO: 2 - DNA, 20 bp, Artificial Sequence
  Synthesized
gtcactccagggcgtacaat

SEQ ID NO: 3 - DNA, 18 bp, Artificial Sequence
  Synthesized
aacgaccccttcattgac

SEQ ID NO: 4 - DNA, 19 bp, Artificial Sequence
  Synthesized
tccacgacatactcagcac

SEQ ID NO: 5 - RNA, 19 nt, Artificial Sequence
  Synthesized
caaacuucaugaucaauca

SEQ ID NO: 6 - RNA, 19 nt, Artificial Sequence
  Synthesized
ugauugaucaugaaguuug

SEQ ID NO: 7 - RNA, 19 nt, Artificial Sequence
  Synthesized
uucuccgaacgugucacgu

SEQ ID NO: 8 - RNA, 19 nt, Artificial Sequence
  Synthesized
acgugacacguucggagaa

(19) United States
(12) Patent Application Publication
(10) Pub. No.: US 2018/0348222 A1
(43) Pub. Date: Dec. 6, 2018

(54) BUFADIENOLIDE FOR TREATMENT OF NON-SMALL CELL LUNG CANCER

(71) Applicant: Macau University of Science and Technology, Taipa (MO)

(72) Inventors: Liang Liu, Taipa (MO); Lai-Han Elaine Leung, Taipa (MO); Xiao-Jun Yao, Taipa (MO); Run-Ze Li, Taipa (MO); Xing-Xing Fan, Taipa (MO)

(21) Appl. No.: 15/610,709

(22) Filed: Jun. 1, 2017

(51) Int. Cl. G01N 33/574; A61K 31/56; A61K 31/585

(52) U.S. Cl. CPC G01N 33/57423; A61K 31/56; C07J 19/00; G01N 33/57492; A61K 31/585

(57) ABSTRACT

A method of treating a subject, in particular a human, suffering from non-small cell lung cancer includes administering a bufadienolide to the subject. A method of inhibiting the proliferation and inducing the cell death of non-small cell lung cancer cells, a method of inhibiting the Epidermal growth factor receptor (EGFR) kinase activity in non-small cell lung cancer cells harboring an abnormality in the EGFR gene, and a method of inhibiting Na⁺/K⁺-ATPase in non-small cell lung cancer cells includes contacting those cells with a bufadienolide. Proscillaridin A as bufadienolide, with the structure of Formula (III) has advantageously high cytotoxicity against EGFR-dependent non-small cell lung cancer at nano-molar levels while having low toxicity to normal lung cells.

### SEQUENCE LISTING

The Sequence Listing file entitled “sequencelisting” having a size of 1,690 bytes and a creation date of 31 May 2017 that was filed with the patent application is incorporated herein by reference in its entirety.

### TECHNICAL FIELD

The present invention relates to a method of treating a subject, in particular a human, suffering from non-small cell lung cancer. The non-small cell lung cancer is especially preferably, but not exclusively, an Epidermal growth factor receptor (EGFR)-dependent non-small cell lung cancer. Said method comprises a step of administering a bufadienolide to the subject. The present invention further provides a method of inhibiting the proliferation and inducing the cell death of non-small cell lung cancer cells, a method of inhibiting the EGFR kinase activity in non-small cell lung cancer cells harboring an abnormality in the EGFR gene and a method of inhibiting Na⁺/K⁺-ATPase in non-small cell lung cancer cells comprising contacting said cells with a bufadienolide.

### BACKGROUND OF INVENTION

Cancer has become the most common disease causing death in China. Carcinoma of the lung has the highest incidence and mortality rates amongst all malignancies. Lung cancer has an incidence of over 1.6 million cases per year accounting for 13% of all new cancer diagnoses and 1.4 million deaths per year accounting for 18% of all cancer-related deaths. Among the various types of lung cancers, non-small cell lung cancer (NSCLC) represents 80 to 85% of all cases and more than 70% are diagnosed as unresectable advanced disease. Although a lot of medical intervention methods were put forward, the prognosis for NSCLC patients remains poor, with the latest 5-year overall survival (OS) rate of 18% of all stages. Up to data, the main strategy to treat the advanced NSCLC has been the direct inhibition of tumor cell growth by cytotoxic agents or targeted small-molecule inhibitors, namely the personalized therapy. In view of the NSCLC driver mutations, including those of EGFR, HER2, KRAS, BRAF, AKT1, ROS1 and ALK, Epidermal growth factor receptor (EGFR) tyrosine kinase inhibitor (TKI) was first administered to NSCLC patients as the personalized therapy which has a response rate higher than 70% and can prolong progression. However these inhibitors, like Iressa, will eventually lose their efficacy due to mutations associated with drug-resistance and, thus, an inevitable relapse.

Sodium potassium pump (Na⁺/K⁺ ATPase) is a transmembrane protein complex functioning as a key energy-consuming pump to maintain ionic and osmotic balance found in all higher eukaryotes. Initiating from the early studies of inhibition of cancer growth in cardiac patients taking cardiac glycosides (Crambert, G. et al., The Journal of biological chemistry 275 (2000) 1976-1986), e.g., a study showed fewer cases of leukemia in a group of patients treated with digitoxin compared to the control group, a potential anticancer effect has been assumed (Jorgensen, P.L., Physiology 65 (2003) 817-849). Cardiac glycosides as secondary metabolites are natural Na⁺/K⁺ ATPase inhibitors (Wang, H. Journal of Biological Chemistry 279 (2004) 17250-17259, Nesher, M., Life Sciences 80 (2007) 2093-2107) and used to cure the congestive heart failure and in atrial arrhythmias for over 200 years. Recently, cytotoxic effects of cardiac glycosides against different cancers have been investigated (Gheorghiade, M. Circulation 113 (2006) 2556-2564, Haux, J., Deutsche Zeitschrift Fur Onkologie 32 (2000) 11-16, Frese, S. Cancer Research 66 (2006) 5867-5874, Haux, J., BMC cancer 1 (2001) 11). Thus, there remains a strong demand for new treatment options for treating NSCLC. In particular, as the efficacy of EGFR inhibitors in EGFR-dependent NSCLC is limited, further potent treatment options for treating EGFR-dependent NSCLC are urgently required. As usual, it would generally be desirable to have treatment options with reduced risk for side effects and interactions based on compounds which can be prepared in a cost-effective way. Usually, plants and respective ingredients in plants might be suitable to provide such advantageous properties.

### SUMMARY OF INVENTION

The present invention in a first aspect relates to a method of treating a subject, in particular a human, suffering from lung cancer. Said method comprises a step of administering an effective amount of a bufadienolide to the subject. The bufadienolide of the present invention comprises and in particular has a structure of Formula (I):

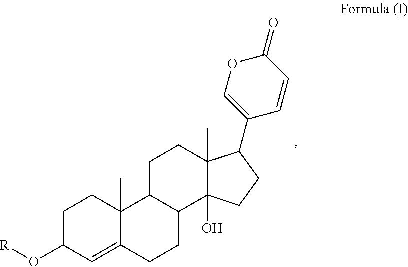

R is H or a glycoside moiety of 1 to 6 sugar residues or is —H.

The bufadienolide preferably comprises and in particular has a structure of Formula (II):

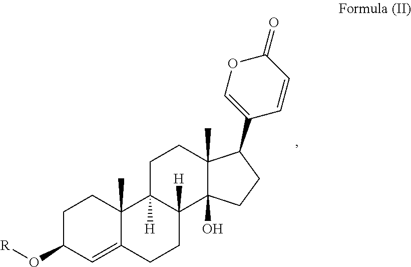

wherein R is as defined above.

The glycoside moiety which can form R may in particular comprise 1 to 3 sugar residues selected from rhamnose and/or glucose such as L-rhamnose and one or two D-glucose residues linked by glycosidic bond such as α-L-Rha(1→), β-D-Glc-(1→4)-α-L-Rha(1→) or β-D-Glc-(1→4)-β-D-Glc-(1→4)- α-L-Rha(1→), i.e. R can be selected from:

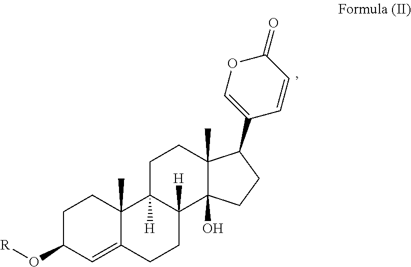

### or —H.

The glycoside moiety which can form R is in particular a monosaccharide, i.e. has one sugar residue, in particular it is α-L-Rha(1→), i.e. the bufadienolide can comprise and in particular has a structure of Formula (III):

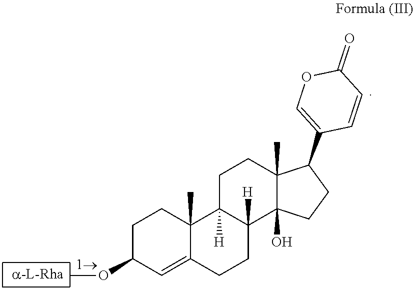

The disease is a NSCLC. The NSCLC is in particular an EGFR-dependent NSCLC, in particular with at least one mutation selected from E746-A750del deletion in exon 19 and/or T790M substitution in exon 20.

The present invention in another aspect refers to a method of inhibiting the proliferation and inducing the cell death of non-small cell lung cancer cells. Said method comprises a step of contacting said cells with an effective amount of a bufadienolide that comprises and in particular has a structure of Formula (I):

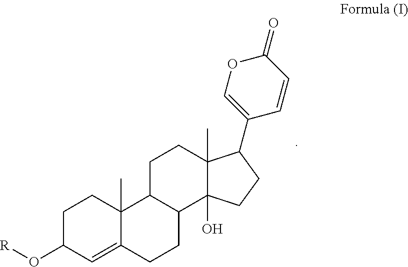

R is H or a glycoside moiety of 1 to 6 sugar residues or is —H.

The present invention provides in another aspect a method of inhibiting the EGFR kinase activity in non-small cell lung cancer cells harboring an abnormality in the EGFR gene. Said method comprises a step of contacting said cells with an effective amount of a bufadienolide that comprises and in particular has a structure of Formula (I):

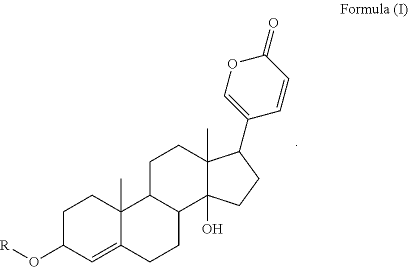

R is H or a glycoside moiety of 1 to 6 sugar residues or is —H.

The abnormality in EGFR gene in particular means at least one of an E746-A750del deletion in exon 19 and/or T790M substitution in exon 20.

In another aspect, the present invention relates to a method of inhibiting Na⁺/K⁺-ATPase in non-small cell lung cancer cells. Said method comprises a step of contacting said cells with an effective amount of a bufadienolide that comprises and in particular has a structure of Formula (I):

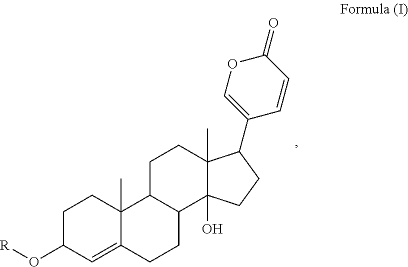

wherein R is H or a glycoside moiety of 1 to 6 sugar residues or is —H.

The inventors in particular unexpectedly found that Proscillaridin A as bufadienolide that has a structure of Formula (III) has advantageously high cytotoxicity against NSCLC cells with the highest cell cytotoxicity in EGFR-dependent NSCLC at nano-molar levels while having low toxicity in normal lung cells representing the first report of Proscillaridin A in NSCLC as specific subtype of lung cancer and in EGFR-dependent NSCLC, either. Moreover, Proscillaridin A proved to inhibit EGFR signaling in EGFR mutant cells but not EGFR wild type cells. In addition to this, the inventors found that Proscillaridin A inhibits Na⁺/K⁺ ATPase and elevates Ca²⁺ level in NSCLC cells, then activates AMPK pathway and downregulates phosphorylation of ACC and mTOR. At last, Proscillaridin A proved to increase Death Receptor 4 expression and down regulates its suppressor NF-κB. Altogether, these results suggest that Proscillaridin A is a highly promising candidate for treatment of NSCLC, in particular of EGFR-dependent NSCLC.

Those skilled in the art will appreciate that the invention described herein is susceptible to variations and modifications other than those specifically described. The invention includes all such variations and modifications. The invention also includes all steps and features referred to or indicated in the specification, individually or collectively, and any and all combinations of the steps or features.

Other features and aspects of the invention will become apparent by consideration of the following detailed description and accompanying drawings.

### BRIEF DESCRIPTION OF DRAWINGS

FIGS. 1A through 1G show dose response curves of Proscillaridin A as bufadienolide having Formula (III) on NSCLC cell lines and CCD19-LU normal lung fibroblast cell line, thus, confirming the selectivity on EGFR-dependent NSCLC cells. Results were expressed as mean ±S.E. (*p<0.05, **p<0.01, ***p<0.001). FIG. 1A shows the dose response curve of Proscillaridin A on A549 cell line. FIG. 1B shows the dose response curve of Proscillaridin A on H1975 cell line. FIG. 1C shows the dose response curve of Proscillaridin A on HCC827 cell line. FIG. 1E shows the dose response curve of Proscillaridin A on H358 cell line. FIG. 1F shows the dose response curve of Proscillaridin A on HCC78 cell line. FIG. 1G shows the dose response curve of Proscillaridin A on CCD-19-Lu cell line.

FIGS. 2A, 2B, and 2C are Western blot patterns showing the inhibition of the EGFR activation and confirming that Proscillaridin A specifically inhibited the phosphorylation of tyrosine residue 1173 on EGFR in EGFR-dependent NSCLC cells. In contrast, Proscillaridin A had no effect on EGFR in EGFR wild-type NSCLC cells A549. FIG. 2A refers to the Western blot pattern of H1975 treated with different concentrations of Proscillaridin A. FIG. 2B refers to the Western blot pattern of HCC827 treated with different concentrations of Proscillaridin A. FIG. 2C refers to the Western blot pattern of A549 treated with different concentrations of Proscillaridin A.

FIG. 3 shows pictures of the colony formation inhibition of different NSCLC cells by Proscillaridin A. Data were shown as representative photomicrographs after treatment with Proscillaridin A at different concentration.

FIGS. 4A through 4D refer to the induction of apoptosis by Proscillaridin A in A549 cell line. FIG. 4A shows the morphological changes of A549 cells after treatment with Proscillaridin A (“P.A”). FIG. 4B shows flow cytometry patterns of the apoptosis level after treatment with Proscillaridin A. FIG. 4C is a bar chart showing the apoptosis level after treatment with Proscillaridin A (“P.A”). FIG. 4D refers to Western blot patterns showing PARP, Caspase-7, Caspase-9 and BAX and their cleavage and activation by Proscillaridin A and Bcl-2 and its down-regulation and the inhibition of the AKT activation (*p<0.05,**p<0.01, ***p<0.001).

FIGS. 5A through 5D refer to the induction of apoptosis by Proscillaridin A in H1975 cell line. FIG. 5A shows the morphological changes of H1975 cells after treatment with Proscillaridin A. FIG. 5B shows flow cytometry patterns of the apoptosis level after treatment with Proscillaridin A. FIG. 5C is a bar chart showing the apoptosis level after treatment with Proscillaridin A. FIG. 5D refers to Western blot patterns showing PARP, Caspase-7, Caspase-9 and BAX and their cleavage and activation by Proscillaridin A and Bcl-2 and its down-regulation and the inhibition of the AKT activation (*p<0.05,**p<0.01, ***p<0.001).

FIGS. 6A through 6D refer to the induction of apoptosis by Proscillaridin A in HCC827 cell line. Proscillaridin A significantly induced apoptosis. FIG. 6A shows the morphological changes of HCC827 cells after treatment with Proscillaridin A. FIG. 6B shows flow cytometry patterns of the apoptosis level after treatment with Proscillaridin A. FIG. 6C is a bar chart showing the apoptosis level after treatment with Proscillaridin A. FIG. 6D refers to Western blot patterns showing PARP, Caspase-7, Caspase-9 and BAX and their cleavage and activation by Proscillaridin A and Bcl-2 and its down-regulation and the inhibition of the AKT activation (*p <0.05,**p<0.01, ***p<0.001).

FIGS. 7A through 7E refer to the inhibition of the Na⁺/K⁺ ATPase and the regulation of the Ca²⁺ level in EGFR-dependent NSCLC cells. FIG. 7A is a graph confirming that Proscillaridin A significantly inhibited the Na⁺/K⁺ ATPase and its IC₅₀ was measured by In Vitro Na⁺/K⁺ ATPase Assay. As shown in FIG. 7B, Proscillaridin A elevated the Ca²⁺ level after 6 h treatment in A549 and H1975 cell lines. FIG. 7C showing flow cytometry patterns confirms that the increase of intracellular Ca²⁺ level was required for Proscillaridin A to induce apoptosis. Calcium chelator (BM) remarkably inhibited the apoptosis induced by Proscillaridin A (*p<0.05,**p<0.01, ***p<0.0001)). FIG. 7D are bar charts showing the Fluo-3 fluorescence under the treatment with different concentrations of Proscillaridin A compared with an untreated control group. FIG. 7E are bar charts showing the percentage of apoptotic cells under the treatment with Proscillaridin A and with Proscillaridin A and BM compared with an untreated control group.

FIGS. 8A through 8F show the activation of the AMPK pathway by Proscillaridin A. FIG. 8A shows Western Blot patterns and confirms that Proscillaridin A increases the phosphorylation of AMPK and its downstream target ACC. FIG. 8B shows flow cytometry patterns and confirms that the inhibition of AMPK by compound C can partially rescue the cells from apoptosis. FIG. 8C and 8D are bar charts showing the percentage of apoptotic cells under treatment with Proscillaridin A and with Proscillaridin A and the compound C. FIG. 8E refers to flow cytometry patterns showing that the JNK inhibitor could weaken apoptosis in both cell lines. FIG. 8F is a bar chart showing the percentage of apoptotic cells under treatment with Proscillaridin A and with Proscillaridin A and the JNK inhibitor.

FIGS. 9A through 9F refer to the increase of the Death Receptor 4 (DR4) expression by Proscillaridin A. FIG. 9A show the pattern obtained after Regular and FIG. 9B after quantitative RT-PCR carried out to determine the expression of DR4 expression after being treated with Proscillaridin A for 12 h. FIG. 9C shows flow cytometry patterns of A549 cells transfected with si-DR4 and treated with or without Proscillaridin A for 18 h. Cells were collected and analyzed by flow cytometry with PI and ANNEXIN V. FIG. 9D is a bar chart showing the percentage of apoptotic cells after the treatment and in the control group. FIG. 9E and 9F are Western blot patterns showing that the activation of NF-κB pathway was inhibited by Proscillaridin A and that the inhibition of the phosphorylation of NF-κB by Proscillaridin A did not change after the knock down of DR4 translation.

FIG. 10A and 10B illustrate the assumed cell model and mechanism of Proscillaridin A in EGFR wild-type (WT) cells (FIG. 10A) and EGFR-dependent (EGFR mutant) cells (FIG. 10B).

### DETAILED DESCRIPTION OF INVENTION

Unless otherwise defined, all technical terms used herein have the same meaning as commonly understood by one skilled in the art to which the invention belongs.

As used herein, “comprising” means including the following elements but not excluding others. “Essentially consisting of” means that the material consists of the respective element along with usually and unavoidable impurities such as side products and components usually resulting from the respective preparation or method for obtaining the material such as traces of further components or solvents. As used herein, the forms “a,” “an,” and “the,” are intended to include the singular and plural forms unless the context clearly indicates otherwise. The terms “optional” or “optionally” means that the described circumstance may or may not occur so that the invention includes instances where the circumstance occurs and instances where it does not occur.

The present invention in a first aspect relates to a method of treating a subject suffering from lung cancer. Said method comprises a step of administering an effective amount of a bufadienolide to the subject.

Bufadienolides are known as compounds present in various plants. They are based on or derived from the following bufadienolide-type basic structure having a C₂₄ steroid structure:

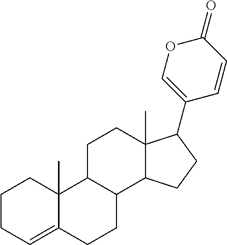 [SMILES: CC12CCCC=C1CCC1C2CCC2(C)C(c3ccc(=O)oc3)CCC12]

The bufadienolide of the present invention comprises and preferably has a structure of Formula (I):

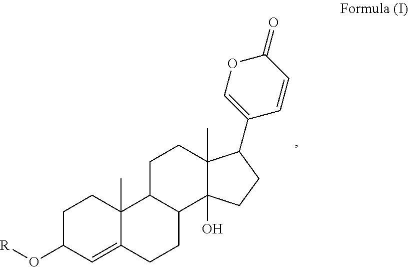

R is H or a glycoside moiety of 1 to 6 sugar residues or is —H. Said structure may optionally be further modified by glycosylation. “Glycosylation” means presence of at least one further glycoside moiety, i.e. one or more sugar residues attached to the OH-group in the structure of Formula (I). Further, it encompasses any solvates or anhydrates of the structure of Formula (I).

In preferred embodiments of the present invention, R is a glycoside moiety of 1 to 3 sugar residues or is —H. The sugar residues are preferably selected from D-rhamnose and/or D-glucose.

The bufadienolide preferably comprises and more preferably has a structure of Formula (II):

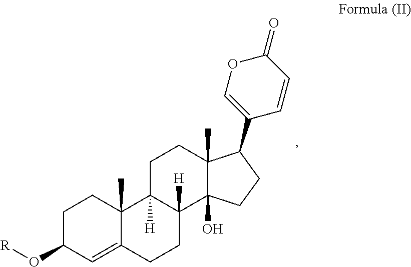

wherein R is as defined above.

The term “glycoside moiety” used herein refers to a moiety formed by optionally substituted monosaccharides. The glycoside moiety has 1 to 6 sugar residues, i.e. can be a mono-, di- or oligosaccharide moiety, for example, formed by one or more of rhamnose and/or glucose. A disaccharide moiety is in particular formed by two monosaccharides linked by glycosidic bond. An oligosaccharide moiety is in particular formed by three or more monosaccharides linked by glycosidic bond. The monosaccharides in the glycoside moiety may be present in different diasteromeric forms, in particular a or p anomers and D or L isomers. The term “glycosidic bond” is a type of chemical bond and covalent linkage formed between the anomeric hydroxyl group of a monosaccharide and the hydroxyl group of another monosaccharide.

The glycoside moiety which can form R in particular comprises 1 to 3 sugar residues selected from rhamnose and/or glucose, further preferred L-rhamnose and/or D-glucose. For example, it can be selected from L-rhamnose and one or two D-glucose residues linked by glycosidic bond such as α-L-Rha(1→), β-D-Glc-(1→4)-α-L-Rha(1→) or β-D-Glc-(1→4)-β-D-Glc-(1→4)- α-L-Rha(1→), i.e. R can be selected from:

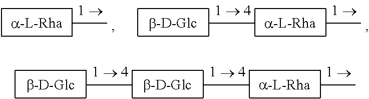

or —H.

The glycoside moiety which can form R is preferably a monosaccharide, i.e. has one sugar residue, in particular it is α-L-Rha(1→), i.e. the bufadienolide can comprise and most preferably can be expressed with Formula (IIIa) and further preferred with Formula (III):

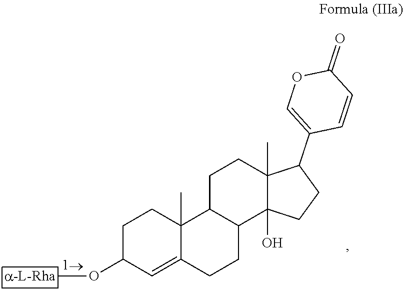

further preferred

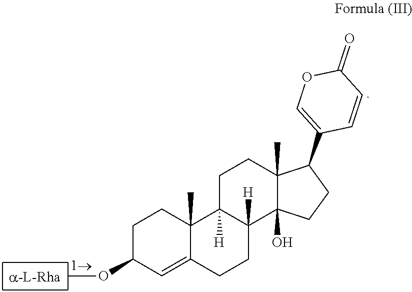

The bufadienolide having a structure of Formula (III) is also known as Proscillaridin A, which can, for example, be obtainable from plants of the genus Scilla and from Drimia maritima (Scilla maritima) and is also commercially available with appropriate purity.

The disease is NSCLC. The terms “cancer” and “cancerous” describe a physiological condition in subjects in which a population of cells are characterized by unregulated malignant (cancerous) cell growth. The cancer is, in particular, an adenocarcinoma.

The NSCLC is preferably an EGFR-dependent NSCLC. The term “EGFR-dependent” as used herein refers to a cancer comprising cancer cells harboring an abnormality in the EGFR gene. An abnormality in the EGFR gene results from a mutation such as due to a substitution, in particular missense substitution, insertion or deletion within the exons 18 to 21 encoding a portion of the EGFR kinase domain, which usually results in an increased kinase activity of EGFR, leading to hyperactivation of downstream pro-survival signaling pathways. In particular, the mutations comprise at least one of an exon 19 deletion or substitution, exon 20 insertion or substitution and/or an exon 21 substitution, in particular at least one of exon 19 deletion and/or an exon 20 substitution. In more preferred embodiments, the abnormality in EGFR gene means at least one mutation selected from E746-A750del deletion in exon 19, L747S substitution in exon 19, D761Y substitution in exon 19, T790M substitution in exon 20, D770_N771 insertion in exon 20, V769L substitution in exon 20, S7681 substitution in exon 20, T854A substitution in exon 21, L858R substitution in exon 21 and/or A871E substitution in exon 21, in particular at least one mutation selected from E746-A750del in exon 19 and/or T790M substitution in exon 20.

Preferably, the abnormality in EGFR gene is associated with a detectable increase in EGFR kinase activity. An “increased kinase activity” of EGFR kinase means an expression of EGFR or an EGFR kinase activity, which is at least 5% and preferably at least 10% and further preferred at least 30% higher compared to a control group, i.e. non-cancerous cells or cancerous cells without abnormality in the EGFR gene. The skilled person is aware of suitable methods for determining EGFR kinase expression or activity such as with immunosorbent assays like with commercially available kits usually with ELISA-based measurement. EGFR expression can be measured, for example, by flow cytometry, real-time PCR, and Western blotting.

Presence of an EGFR mutation can be confirmed by respective molecular biological methods, wherein several methods are known to the skilled person. Such tests are commonly performed using DNA or RNA collected from biological samples, e.g., tissue biopsies, and can be conducted by a variety of methods including, but not limited to, sequence-specific PCR, direct DNA sequencing, hybridization with allele-specific probes, enzymatic mutation detection, chemical cleavage of mismatches or mass spectrometry. I.e. EGFR-dependent NSCLC is in particular considered for being present, if at least one of the above methods reveals an EGFR mutation in the cancer cells of the NSCLC.

The EGFR-dependent NSCLC can have an intrinsic or acquired resistance against at least one EGFR inhibitor, in particular against at least one of gefitinib, erlotinib and/or afatinib, more preferably an intrinsic or acquired resistance at least against gefitinib. This means that the cells with EGFR gene abnormality can have an intrinsic or acquired resistance against at least one EGFR inhibitor, in particular at least against gefitinib.

Such resistance can be caused by or follow from the EGFR mutation as such, for example due to insertions or substitutions in exon 20, in particular due to a T790M substitution in exon 20, so that the EGFR inhibitors cannot provide therapeutic advantages. An acquired resistance can also follow from, for example, MET gene amplification encoding MET receptor tyrosine kinase and/or fibroblast growth factor 2 (FGF2) and FGF receptor 1 (FGFR1) induction, mitogen-activated protein kinase 1 (MAPK1) amplification, mutations in downstream effector proteins to EGFR, epithelial-to-mesenchymal transition and small-cell transformation. Such EGFR inhibitor resistance can be detected in a subject, tissue, or cell by administering to a subject, tissue or cell an EGFR inhibitor and determining its activity such as the induction of cell death, the inhibition of the proliferation of cancer cells or the activation of EGFR such as one or more of the phosphorylation of EGFR or its signaling proteins like p-AKT compared to a control sample of the same cells or tissue without treatment with the EGFR inhibitor and/or compared to a reference sample, namely cells or tissue of the same cell or tissue type or a subject that do not have EGFR inhibitor resistance. This can be carried out by methods known to the skilled person like phosphoprotein assays, cell viability measurement with MTT assays or Western Blotting or the like.

The term “subject” used herein refers to a living organism and can include but is not limited to a human and an animal. The subject is preferably a mammal, preferably a human. The bufadienolide may be administered by an oral or parenteral route to the subject, preferably a human.

The expression “effective amount” generally denotes an amount sufficient to produce therapeutically desirable results, wherein the exact nature of the result varies depending on the specific disorder which is treated. When the disorder is cancer, the result is usually an inhibition or suppression of the proliferation of the cancer cells, a reduction of cancerous cells or the amelioration of symptoms related to the cancer cells, in particular inhibition of the proliferation of the cancer cells or induction of cell death, i.e. apoptosis of the cancer cells.

The effective amount of the bufadienolide of the present invention may depend on the species, body weight, age and individual conditions of the subject and can be determined by standard procedures such as with cell cultures or experimental animals. A concentration of the bufadienolide such as a bufadienolide of Formula (III) may, for example, be at least about 10 nM, in particular at least about 25 nM, for example, about 50 nM.

The bufadienolide may be administered in form of a pharmaceutical composition comprising the bufadienolide and at least one pharmaceutically tolerable excipient such as one or more of a diluent, a filler, a binder, a disintegrant, a lubricant, a coloring agent, a surfactant and a preservative. The pharmaceutical composition can be present in solid, semisolid or liquid form. The pharmaceutical composition may comprise further pharmaceutical effective ingredients such as therapeutic compounds which are used for treating NSCLC.

The skilled person is able to select suitable pharmaceutically tolerable excipients depending on the form of the pharmaceutical composition and is aware of methods for manufacturing pharmaceutical compositions as well as able to select a suitable method for preparing the pharmaceutical composition depending on the kind of pharmaceutically tolerable excipients and the form of the pharmaceutical composition. The pharmaceutical composition according to the invention may be administered by an oral or parenteral route to a subject, preferably a human.

In an embodiment, the bufadienolide can be used as a single compound for treating the subject.

In other embodiments, the bufadienolide is administered in combination with other therapeutically effective treatments such as one or more of:
other chemotherapeutic compounds which are used for treating NSCLC; and/or
radiation therapy.

The compound of Formula (I) may be used in combination with other chemotherapeutic compounds used for treating NSCLC. Such therapeutic compounds may include one or more of an angiogenesis inhibitor, an EGFR inhibitor, an anaplastic lymphoma kinase (ALK) inhibitor, a BRAF/MEK inhibitor or a cytotoxic agent including, for example, a topoisomerase-II inhibitor, an anthracycline, a coordination complex of platinum, a taxane, a vinca alkaloid or derivative thereof, a topoisomerase-I inhibitor and a nucleotide analog or precursor analog. The bufadienolide of Formula (I) can be prepared by suitable methods, such as by chemical synthesis or by extraction from plant materials.

The method of the present invention may further include steps carried out before administering the bufadienolide of Formula (I), such as the bufadienolide of Formula (IIa), to the subject comprising:
obtaining a sample, in particular cancer cells, from the subject;
testing said sample for the EGFR kinase activity and/or identifying at least one EGFR mutation as abnormality in the EGFR gene;
optionally correlating the EGFR kinase activity and/or abnormality in the EGFR gene with outcome and if conditions are met, administrating the bufadienolide of Formula (I), in particular of Formula (III), to said subject.

The present invention in another aspect refers to a method of inhibiting the proliferation and inducing the cell death of non-small cell lung cancer cells. Said method comprises a step of contacting said cells with an effective amount of a bufadienolide that comprises and in particular has a structure of Formula (I):

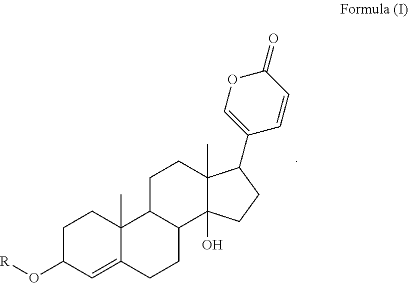

R is H or a glycoside moiety of 1 to 6 sugar residues or is —H.

The step of contacting the cancer cells with the bufadienolide of the present invention, in particular comprising a structure of Formula (III), may be carried out by applying an incubation solution comprising the bufadienolide to said cells which incubation solution may further comprise suitable excipients such as buffers or a suitable growth medium. In such embodiment of the present invention, the cells are taken as a cell probe from the cancer cells of a subject such as an animal or human, in particular a human. In other preferred embodiments of the present invention, the step of contacting the non-small cell lung cancer cells is carried out by administering the bufadienolide of the present invention to a subject, in particular a human, which subject comprises the non-small cell lung cancer cells.

Inducing cells death includes inducing cell death by apoptosis or other mechanisms of cell death, in particular by apoptosis. The induction of apoptosis can be determined by means of microscopic and flow cytometric analysis or determination of the activation and cleavage, respectively, of PARP, Caspase-7 and Caspase-9 or activation of pro-survival regulators such as AKT and pro-apoptotic proteins such as BAX and anti-apoptotic proteins such as BCL-2 such as by means of Western Blotting. The percentage of apoptotic cells is increased, in particular significantly increased, i.e. a statistical significant increase and further preferred increased by at least 5 percentage points compared to an untreated reference control. “Statistically significant” means a result that generally is at least two standard deviations above or below the mean of at least three separate determinations of a control and/or that is statistically significant as determined by Student's t-test or other art-accepted measures of statistical significance.

The induction of apoptosis by the bufadienolide of the present invention is preferably mediated by one or more, in particular all of:
an inhibition of the Na⁺/K⁺ ATPase and in particular by an elevation of the cellular Ca²⁺ levels, which can be determined by means of an Na⁺/K⁺ ATPase assay with measurement of cellular Ca²⁺ levels by means of a fluorescent dye such as Fluo-3;
activation of Adenosine monophosphate-activated protein kinase (AMPK) which can be determined by means of Western Blotting; and
the expression of Death Receptor 4 (DR4), which can be determined by RT-PCR.

The term “inhibiting proliferation” of a cell includes rendering the cell incapable of growing or dividing or reducing or retarding cell growth or division. The percentage of viable cells which can be used as a measure for cell proliferation determined by means of cell viability assays after contacting the cells with the bufadienolide is preferably decreased, more preferably significantly decreased, i.e. there is a statistically significant decrease. The percentage of viable cells determined by means of cell viability assays after contacting the cells with the bufadienolide is preferably significantly decreased between concentrations of about 10 nM to about 20 nM and at higher concentrations of the bufadienolide. The cell proliferation can further be determined by means of, for example, colony formation assays.

The cytotoxic concentration of the bufadienolide to cause death to 50% of viable cells (CC₅₀) is preferably at most 50 nM, in particular at most 20 nM and further preferred at most 18 nM or at most 16 nM in non-small cell lung cancer cells, in particular in those with an abnormality in the EGFR gene and at least 200 nM, in particular at least 400 nM in normal lung cells (normal lung fibroblast cells). The CC₅₀ of the bufadienolide on non-cancerous lung cells is preferably at least 10 times higher, more preferably 20 times higher than the CC₅₀ on NSCLC cells.

The effective amount of the bufadienolide for contracting the cells is preferably at least about 10 nM, more preferred at least about 12.5 nM, in particular at least about 25 nM and further preferred at least about 50 nM. The effective amount of the bufadienolide for contracting the cells is preferably at most 100 nM.

The cells are preferably contacted with the bufadienolide for at least about 6 h, in particular for at least about 24 h.

The NSCLC cells contacted with the bufadienolide may comprise between 1.0×10³ cells and 1.0×10⁶ cells, in particular about 1.0×10⁵ cells.

The non-small cell lung cancer cells are preferably from an adenocarcinoma. The non-small lung cancer cells in particular have an abnormality in EGFR gene. The abnormality in EGFR gene preferably means at least one of an exon 19 deletion or substitution, exon 20 insertion or substitution and/or an exon 21 substitution, in particular at least one of E746-A750del deletion in exon 19, L747S substitution in exon 19, D761Y substitution in exon 19, T790M substitution in exon 20, D770_N771 insertion in exon 20, V769L substitution in exon 20, S7681 substitution in exon 20, T854A substitution in exon 21, L858R substitution in exon 21 and/or A871E substitution in exon 21, more preferably at least one of E746-A750del deletion in exon 19 and/or T790M substitution in exon 20. The abnormality in the EGFR gene in particular results from at least one of E746-A750del deletion in exon 19 and/or T790M substitution in exon 20.

The non-small cell lung cancer cells with abnormality in EGFR gene can have an intrinsic or acquired resistance against at least one EGFR inhibitor such as selected from at least one of gefitinib, erlotinib and/or afatinib, further preferred an intrinsic or acquired resistance at least against gefitinib.

The bufadienolide comprises and most preferably has in preferred embodiments of the method of inhibiting the proliferation and inducing the cell death of non-small cell lung cancer cells a structure of Formula (IIIa), in particular of Formula (III):

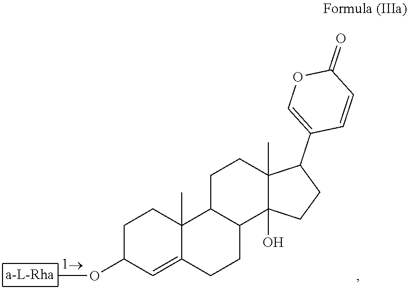 [SMILES: COC1C=C2CCC3C(CCC4(C)C(c5ccc(=O)oc5)CCC34O)C2(C)CC1]

further preferred

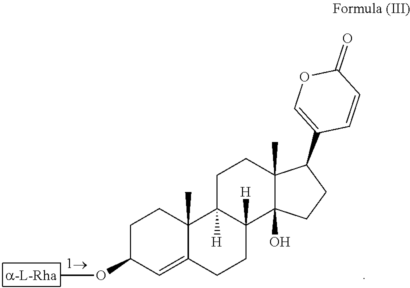

The present invention provides in another aspect a method of inhibiting the EGFR kinase activity in non-small cell lung cancer cells harboring an abnormality in the EGFR gene. Said method comprises a step of contacting said cells with an effective amount of a bufadienolide that comprises and more preferably has a structure of Formula (I):

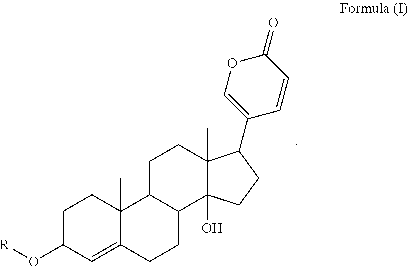

R is H or a glycoside moiety of 1 to 6 sugar residues or is —H.

The step of contacting the cancer cells with the bufadienolide of the present invention, in particular comprising a structure of Formula (III), may be carried out by applying an incubation solution comprising the bufadienolide to said cells which incubation solution may further comprise suitable excipients such as buffers or a suitable growth medium. In such embodiment of the present invention, the cells are taken from a subject such as an animal or human, in particular a human. In other preferred embodiments of the present invention, the step of contacting the non-small cell lung cancer cells is carried out by administering the bufadienolide of the present invention to a subject, in particular a human, which subject comprises the non-small cell lung cancer cells.

The abnormality in EGFR gene preferably means at least one of an exon 19 deletion or substitution, exon 20 insertion or substitution and/or an exon 21 substitution, in particular at least one of E746-A750del deletion in exon 19, L747S substitution in exon 19, D761Y substitution in exon 19, T790M substitution in exon 20, D770_N771 insertion in exon 20, V769L substitution in exon 20, S7681 substitution in exon 20, T854A substitution in exon 21, L858R substitution in exon 21 and/or A871E substitution in exon 21, more preferably at least one of E746-A750del deletion in exon 19 and/or T790M substitution in exon 20. The abnormality in the EGFR gene in particular results from at least one of E746-A750del deletion in exon 19 and/or T790M substitution in exon 20.

The inhibition of the EGFR kinase activity can be determined with assays or for example by determining the autophosphorylation of tyrosine residues like 1173 in EGFR. This can be carried out, for example, by means of Western blotting. Inhibiting EGFR kinase activity is associated with a suppression of anti-apoptotic and growth signaling pathways that are downstream to EGFR.

The non-small cell lung cancer cells with abnormality in EGFR gene are preferably from an adenocarcinoma. The non-small cell lung cancer cells with abnormality in EGFR gene can have an intrinsic or acquired resistance against at least one EGFR inhibitor such as selected from at least one of gefitinib, erlotinib and/or afatinib, further preferred an intrinsic or acquired resistance at least against gefitinib.

The effective amount of the bufadienolide for contracting the cells is preferably at least about 10 nM, further preferred at least about 12.5 nM, in particular at least about 25 nM and further preferred at least about 50 nM. The effective amount of the bufadienolide for contracting the cells is preferably at most 100 nM.

The cells are preferably contacted with the bufadienolide for at least about 6 h, in particular for at least about 18 h.

The bufadienolide comprises and most preferably has in preferred embodiments of the method of inhibiting the EGFR kinase activity a structure of Formula (IIIa), in particular of Formula (III):

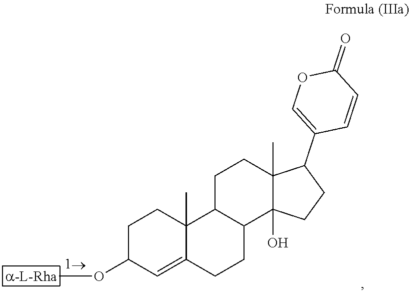

further preferred

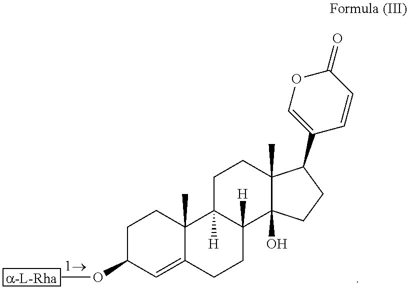

In another aspect, the present invention relates to a method of inhibiting Na⁺/K⁺-ATPase in non-small cell lung cancer cells. Said method comprises a step of contacting said cells with an effective amount of a bufadienolide that comprises and preferably has a structure of Formula (I):

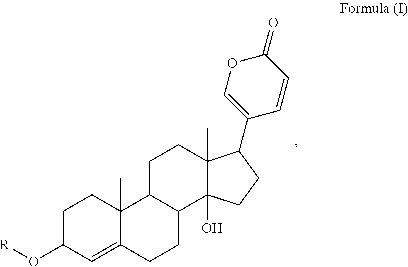

wherein R is H or a glycoside moiety of 1 to 6 sugar residues or is —H.

In particular, the bufadienolide comprises and more preferably has a structure of Formula (IIIa), in particular of Formula (III):

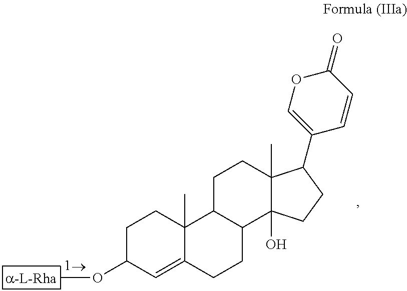 [SMILES: COC1C=C2CCC3C(CCC4(C)C(c5ccc(=O)oc5)CCC34O)C2(C)CC1]

further preferred

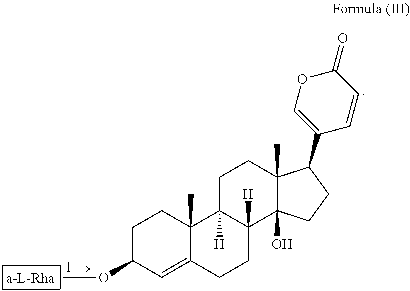

The step of contacting the cancer cells with the bufadienolide of the present invention, in particular comprising a structure of Formula (III), may be carried out by applying an incubation solution comprising the bufadienolide to said cells which incubation solution may further comprise suitable excipients such as buffers or a suitable growth medium. In such embodiment of the present invention, the cells are taken from a subject such as an animal or human, in particular a human. In other preferred embodiments of the present invention, the step of contacting the non-small cell lung cancer cells is carried out by administering the bufadienolide of the present invention to a subject, in particular a human, which subject comprises the non-small cell lung cancer cells.

The inhibition of the Na⁺/K⁺ ATPase is in particular accompanied by an elevation of the cellular Ca²⁺ levels and can be measured by means of a Na⁺/K⁺ ATPase assay with measurement of cellular Ca²⁺ levels by means of a fluorescent dye such as Fluo-3.

The cells are in particular incubated with the bufadienolide of the present invention for at least about 30 min.

### EXAMPLES

### Material and Methods

### Cell Culture and Reagents

Seven lung cancer cell lines (A549, H1975, HCC827, H1819, H2228, H358 and HCC78), and one normal lung cell line (CCD19-LU) were purchased from ATCC (American type culture collection). HCC827 cells are adenocarcinoma cells harboring high level EGFR amplification and an E746-A750del deletion in exon 19. H1975 cells are adenocarcinoma cells harboring L858R substitution in exon 21 and a T790M substitution in exon 20 which is directly associated with resistance against gefitinib. A549 is an NSCLC EGFR wild-type cell line. H2228 is an NSCLC cell line with an EML4-ALK variant. H358 is a NSCLC cell line with KRAS mutation. HCC78 is a NSCLC cell line which expresses the SLC34A2-ROS1 fusion. The lung cancer cell lines were cultivated with RPMI 1640 medium. CCD19-LU were cultivated with MEM medium. Both RPMI 1640 and MEM medium were supplemented with 10% fetal bovine serum (Gibco, Big Cabin, Oklahoma, Me., USA) as well as 100 U/mL penicillin and 100 μg/mL streptomycin (Gibco, Big Cabin, Oklahoma, Me., USA). The cells were cultured in an incubator with 5% CO₂ at 37° C.

Proscillaridin A, i.e. the bufadienolide of Formula (III), was purchased from Sigma (St Louis, Mo., USA). The primary antibodies of β-actin, GAPDH, total/phosphor-EGFR, total/phosphor-AKT, total/phosphor-JNK, total/phosphor-AMPK, total/phosphor-ACC, phosphor-mTOR, IKK-α, Iκ-Bα, phosphor-NFκB-P65, Caspase-9, Caspase-7, BAX and BCL-2 were purchased from Cell Signaling Technology (Danvers, Mass., USA). Fluorescein-conjugated anti-rabbit as secondary antibody was purchased from Odyssey (Belfast, Me., USA).

### MTT Cytotoxicity Assay

Cells were seeded in a 96-well microplate with 3000-5000 cells/well confluence, and put into the incubator overnight for cells adhesion. Different concentrations of drug Proscillaridin A were added with Dimethyl sulfoxide (DMSO) as vehicle control. The microplates were incubated for further 24 h. Each dosage was repeated in triplicate. 10 μL of MTT (5 mg/mL) solution was added to each well. The plate was placed back into the incubator for 4 h. After that, 100 μL of resolved solution (10% SDS and 0.1 mM HCL) was added to each well.

Before dissolving the formazan crystals, the microplate was put back into the incubator for another 4 h. The absorbance of the plate was measured at 570 nm with reference 650 nm by a microplate reader (Tecan, Morrisville, N.C., USA). Cell viability was calculated by percentages of the absorbance of the treatment group divided by the absorbance of untreated group. At least three independent experiments were performed for data analysis and presentation.

### Colony-Formation Assay

Colony-forming assay was performed as previously described. Briefly, About 300 cells were plated into every well of 6 -well plate with 2 ml of culture medium and grown at 37° C. with 5% CO₂. After 48 h culture for cell adherence to the plate, rinsed with fresh medium, Proscillaridin A (with indicated different dosages) was added to the medium. 48 h later, the cells were washed twice with PBS and then incubated in drug-free medium. The medium was changed every 5 days. After culturing for additional 10 to 14 days, the medium was discarded and each dish was washed twice with PBS carefully. The cells were fixed with 3.7% Paraformaldehyde for 20 min and 0.2% crystal violet solution in 10% ethanol for 10 min. Excess stain was removed by washing repeatedly with PBS. All the procedures were done at room temperature. The plates can be stored at room temperature or at +4° C. for several months without any visible fading of the dye.

### Apoptosis Assay

A549, H1975 and HCC827 cells (1×10⁵ cells /well) were seeded in a 6 -well plate for 24 h, and treated with the indicated concentrations of Proscillaridin A for an additional 24 h at 37° C. After indicated hours, the cells were washed by ice-cold 1×PBS once and harvested by trypsination. Then cell were centrifuged, collected and resuspended in ice-cold 1×PBS. After removing the supernatants, cell pellets were re-suspended in 100 μL 1× Annexin-binding buffer. The cells were then double-stained with Annexin-V FITC and PI (100 μg/mL) of 2 μL respectively for 15 min at room temperature in dark. After that, 300 μL 1× Annexin-binding buffer was added. Apoptotic cells were quantitatively counted by a BD Aria III Flow Cytometer (BD Biosciences, San Jose, Calif., USA).

### Western Blot Analysis

After incubation A549, H1975 and HCC827 cells with Proscillaridin A for 18 hours, cells were harvested and washed with cold 1× PBS. Then, cells were lysed with ice-cold RIPA lysis buffer with protease and phosphatase inhibitors added to extract the cell protein extraction. The supernatants were collected by centrifugation at 12,000 g, for 5 min. The quantitation of total protein extraction was measured by Bio-Rad DCTM protein assay kit (Bio-Rad, Philadelphia, Pa., USA). Then 35 μg of protein were loaded and electrophoretically separated on 8% SDS-PAGE gel and then transferred to Nitrocellulose (NC) membrane.

Membranes were blocked with 5% non-fat milk and PBS containing 0.1% Tween-20 (TBST) for 1 h at room temperature. After 1 h, membranes were incubated with primary antibodies (1:1000 dilution) against β-actin, GAPDH, total/phosphor-EGFR, total/phosphor-AKT, total/phosphor-JNK, total/phosphor-AMPK, total/phosphor-ACC, phosphor-mTOR, IKK-α, Iκ-Bα, phosphor-NFκB-P65, Caspase-9, Caspase-7, BAX and Bcl-2 at 4° C. with gently shaking overnight. Membranes were washed with TBST for 3 times (5 min/time), and incubated with secondary fluorescent antibody (1:10000 dilutions) for 1 h at room temperature. Rewashing with TBST for 3 times (15 min/time), the stripes were visualized by LI-COR Odessy scanner (Belfast, Me., USA).

### Na⁺/K⁺ ATPase Enzyme Activity Assay

The enzymatic activity of Na⁺/K⁺ ATPase (purchased from Sigma as lyophilized powder from porcine cerebral cortex) was measured by colorimetric quantification of P, released during ATP hydrolysis. A previously published procedure was adapted with some modifications. Begin with incubating 10 μl of Na⁺/K⁺ ATPase (600 units/m1) with 2.5 μl of KCl/NaCl solution (45 mM KCl and 2 M NaCl) at 37° C. for 30 min with either 5 μl of DMSO (control) or 5 μl of P.A (in different indicated concentrations) in 67.5 μl of buffer (24 mM Tric HCl buffer with 0.68 mM ethylenediaminetetraacetic acid and 6.0 mM magnesium chloride, pH 7.8). ATP (5 μl of 80 mM solution) was then added, and the reaction mixture was incubated again at 37° C. for 15 min. Trichloroacetic acid (30 μl of 100% w/v) was then added to the reaction mixture followed by centrifugation for 5 min. Supernatant (50 μl aliquot) was transferred to a 96 well plated containing 100 μl of Taussky-Shorr reagent. The absorbance at 660 nm was read after incubation at RT for 5 min.

### Measurement of Intracellular Calcium

Changes in intracellular free calcium were measured by a fluorescent dye, Fluo-3 as previously described. Briefly, A549 and H1975 cells were washed twice with culture media after Proscillaridin A treatment (6.25-25 nM) for 6 hours. Then cell suspensions were incubated with 5 μM Fluo-3 at 37° C. for 30 min. After the cells were washed twice with HBSS, the re-suspended cell samples were then subjected to FACS analysis. At least 10,000 events were analyzed.

### RNA Extraction and Quantitative Real-Time PCR

Cells were incubated with Proscillaridin A for 12 hours. Total RNA was then extracted from treated cells using a TRIzol reagent (Invitrogen, Carlsbad, Calif., USA) following the manufacturer's instructions and was used to prepare cDNA. Quantitative real-time PCR was performed with High-productivity Real-Time quantitative PCR ViiA™7 (Life Technologies).

The sequences of PCR primers used were synthesized commercially, and are given in Table 1.

TABLE 1
sequences of PCR primers

| | sequence | SEQ. ID. NO: |
|---|---|---|
| DR4 | 5′-TTGTGTCCACCAGGATCTCA-3′ | SEQ. ID. NO: 1 |
| | and | |
| | 5′-GTCACTCCAGGGCGTACAAT-3′ | SEQ. ID. NO: 2 |
| GAPDH | 5′-AACGACCCCTTCATTGAC-3′ | SEQ. ID. NO: 3 |
| | and | |
| | 5′-TCCACGACATACTCAGCAC-3′ | SEQ. ID. NO: 4 |

The glyceraldehyde 3-phosphatase dehydrogenase (GAPDH) gene was used as the reference gene. All data were means of fold change of triplicate analysis and normalized with those of GAPDH.

### Transfection with Small Interfering RNA

A549 cells were seeded into 6-well plates. After 24 h, cells were transfected with 1 μg DR4 and control small interfering RNA (siRNA) by using 2 μL X-tremeGENE siRNA transfection reagent (Roche Germany) according to the manufacturer's protocols. All the siRNAs were synthesized by GenePharma.

The sequences of siRNAs used are given in Table 2 (sense and antisense, respectively).

TABLE 2
sequences of siRNAs

| | sequence | SEQ. ID. NO: |
|---|---|---|
| siDR4 | 5′-r(CAAACUUCAUGAUCAAUCA)dTdT-3′ and | SEQ. ID. NO: 5 |
| | 5′-r(UGAUUGAUCAUGAAGUUUG)dAdT-3′ | SEQ. ID. NO: 6 |
| control siRNA | 5′-r(UUCUCCGAACGUGUCACGU)dTdT-3′ and | SEQ. ID. NO: 7 |
| | 5′-r(ACGUGACACGUUCGGAGAA)dTdT-3′ | SEQ. ID. NO: 8 |

8 h after transfection, cells were washed with PBS, culture medium was replaced, and cells were stimulated with 12.5 nM Proscillaridin A. Cells were harvested either 18 h after stimulation for determination of apoptosis by flow cytometry.

### Statistical Analysis

All the data were presented as mean ±SD of 3 individual experiments. Differences were analyzed by one-way ANOVA using Graph Prism 5.

### Results and Discussion

### Cytotoxic Effects of Proscillaridin A Towards EGFR-Dependent NSCLC Cells

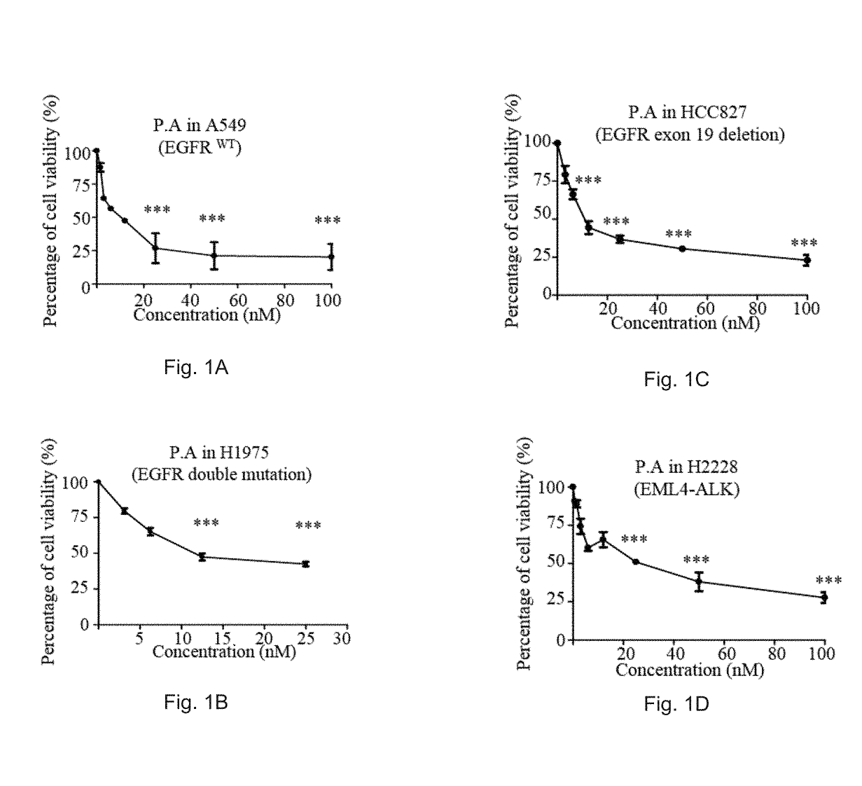

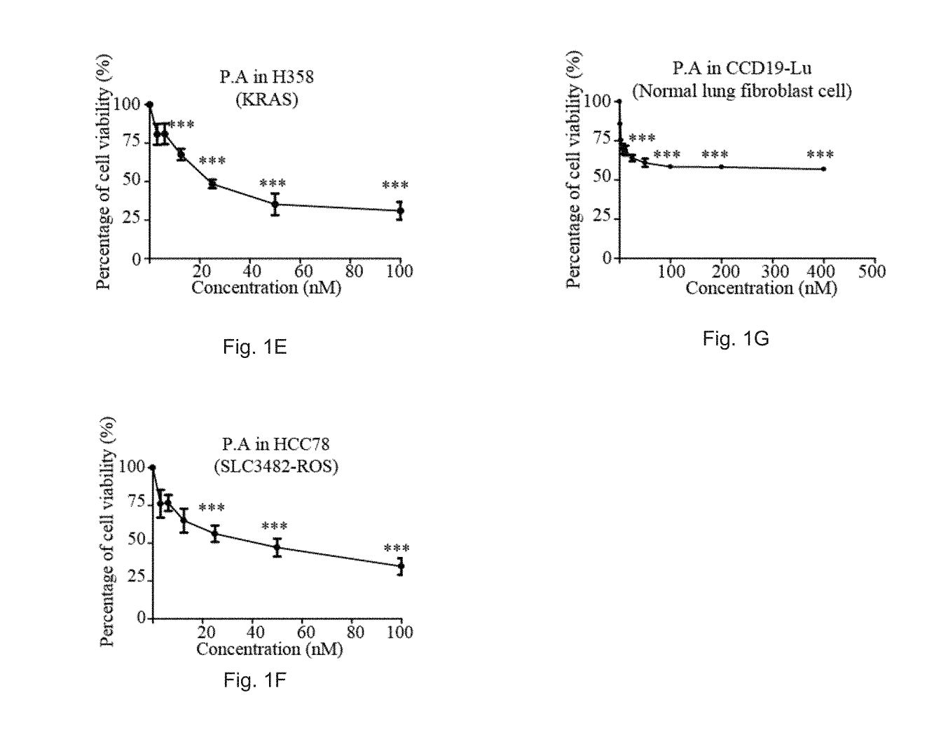

The effect of Proscillaridin A on cell viability was investigated in seven lung cancer cell lines (A549, H1975, HCC827, H1819, H2228, H358 and HCC78), and one normal lung fibroblast cell line (CCD19-LU) (FIG. 1 and Table 3). These NSCLC cell lines have different primary mutations, including EGFR, ALK, KRAS, ROS related mutations, as the main NSCLC driver mutations. After culturing and treating the cells with different concentrations of Proscillaridin A for 24 h, the viability of cells was determined by the means of MTT assay. As shown in the Table 3, P.A was more effective in reducing the growth of EGFR (A549 having the wild type EGFR; H1975 harboring L858R and T790M EGFR double mutant; and HCC827 harboring EGFR exon 19 deletion) related NSCLC cells than the other NSCLC cells. Interestingly, the inhibition of the cell proliferation by Proscillaridin A in normal lung fibroblast cells (CCD19-LU) was the lowest. Even using 30-folds higher concentrations of the CC₅₀ in EGFR-dependent NSCLC cells to treat CCD19-LU, there is still no significant inhibitory effect, implying low toxicity of Proscillaridin A to normal lung fibroblast cells.

TABLE 3
The CC₅₀ value of Proscillaridin A on NSCLC cell lines and CCD19-LU normal lung fibroblast cell line

| | Cell lines | | | | | | |
|---|---|---|---|---|---|---|---|
| | | | | | | | Normal Cell line |
| | NSCLC | NSCLC | NSCLC | NSCLC | NSCLC | NSCLC | CCD19-LU, |
| | HCC827 | A549 | H1975 | H2228 | H358 | HCC78 | Fibroblast cell |
| Mutation | EGFR exon | EGFR | EGFR | EML4 - | KRAS | SLC3482 - | |
| | 19 deletion | wild | double | ALK | | ROS | |
| | | type | mutant | | | | |
| Gefitinib | Sensitive | Sensitive | Resistant | | | | |
| sensitive | | | | | | | |
| CC₅₀ (nM) | 13.9 ± 1.8 | 14.0 ± 2.8 | 15.0 ± 3.6 | 23.6 ± 5.4 | 25.8 ± 4.9 | 37.6 ± 9.9 | >400 |

### EGFR Activation and Induction of Apoptosis in EGFR-Dependent NSCLC Cell Lines and in EGFR Wild Type Cell Lines

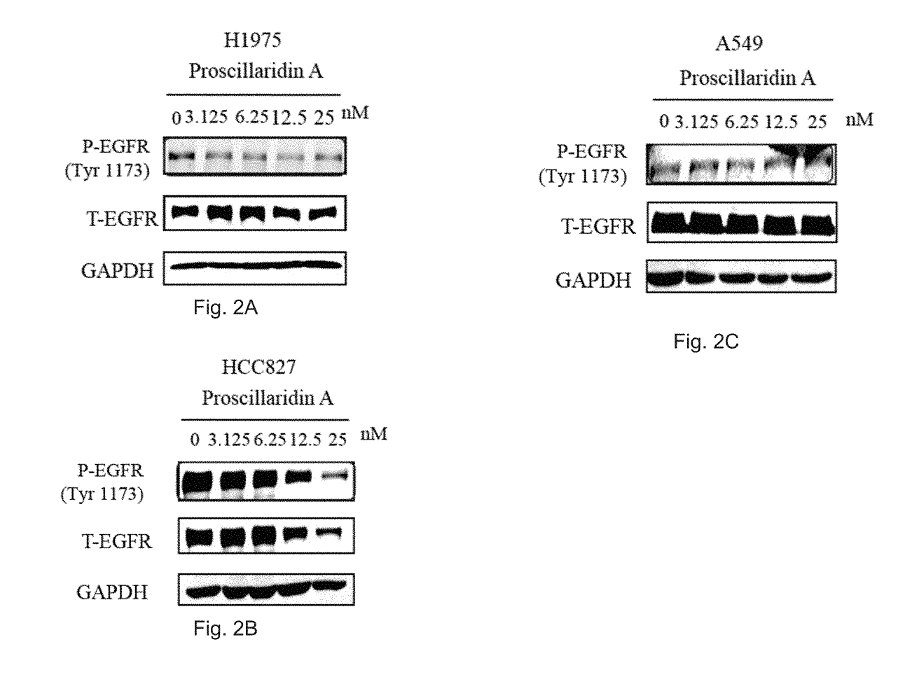

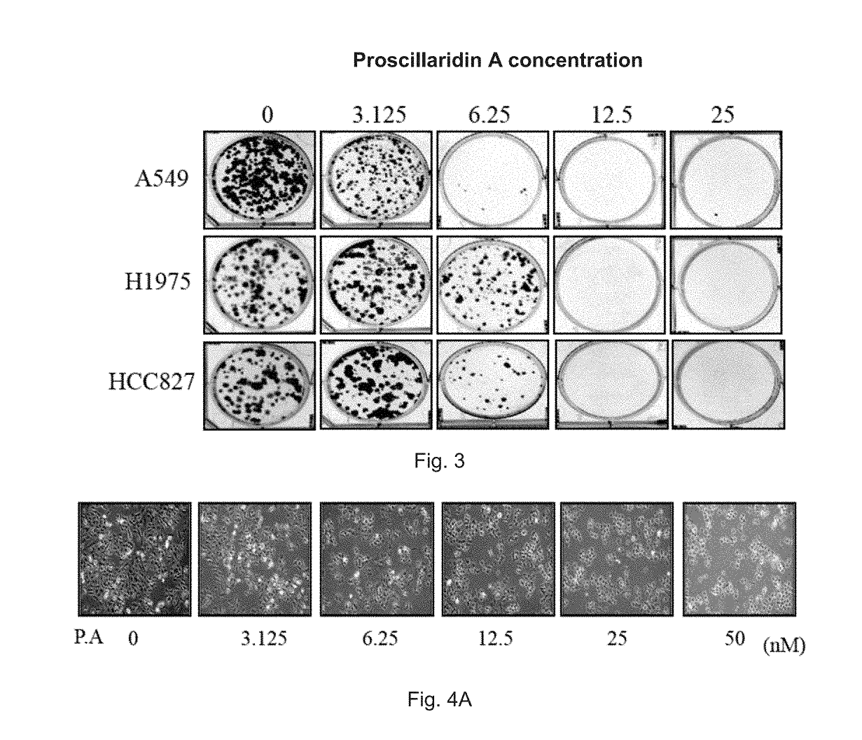

The effect of Proscillaridin A on the activation of EGFR phosphorylation has been evaluated. So the EGFR activation was measured. The results show that Proscillaridin A inhibited the EGFR activation, namely the phosphorylation of tyrosine residue 1173 in EGFR-dependent NSCLC cell lines, H1975 and HCC827 but not in A549, the EGFR wild type (FIG. 2). Further, it has been evaluated whether treatment of Proscillaridin A affects the clonogenic growth in these three cell lines by measuring the colony formation. The results demonstrate that Proscillaridin A can suppress clonogenic growth in all the three EGFR-dependent NSCLC cell lines (FIG. 3).

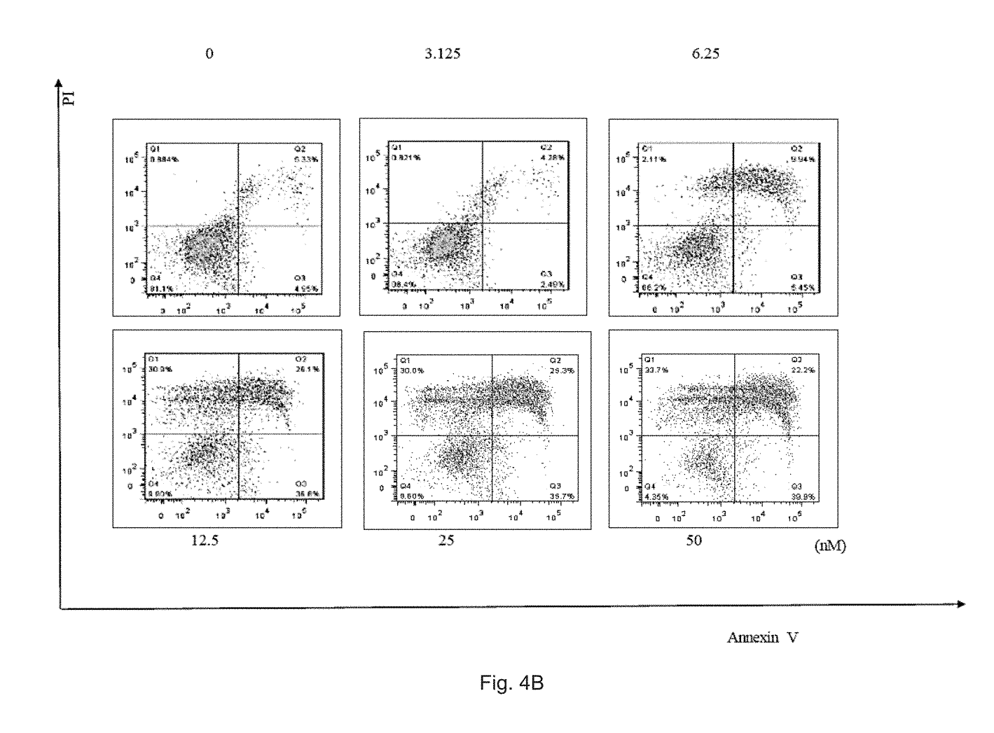

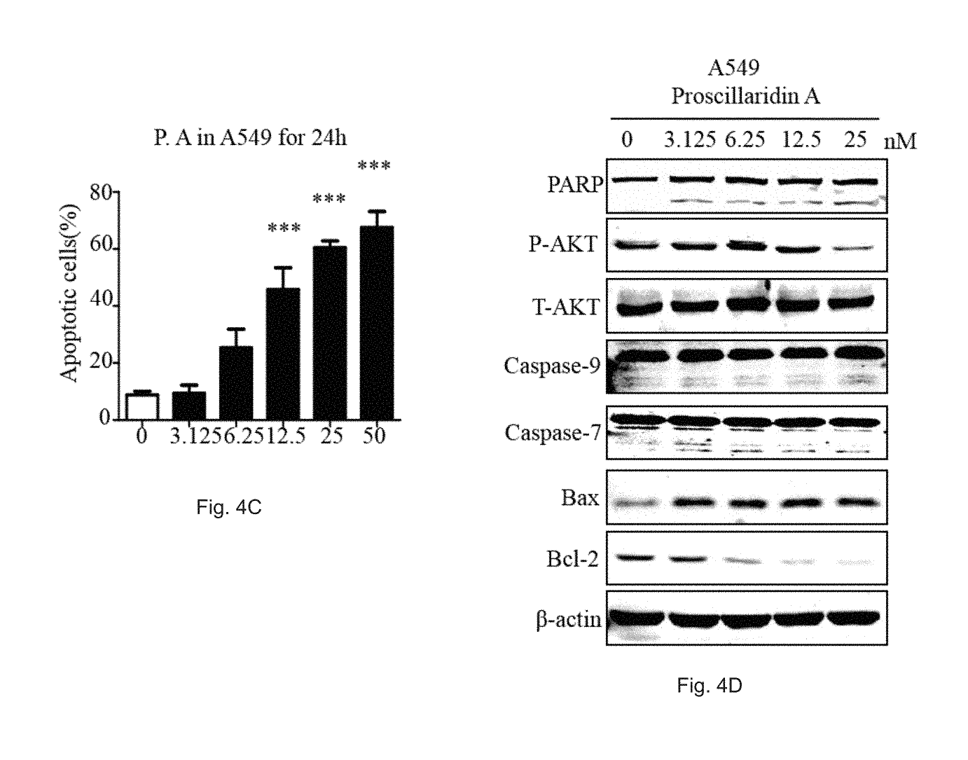

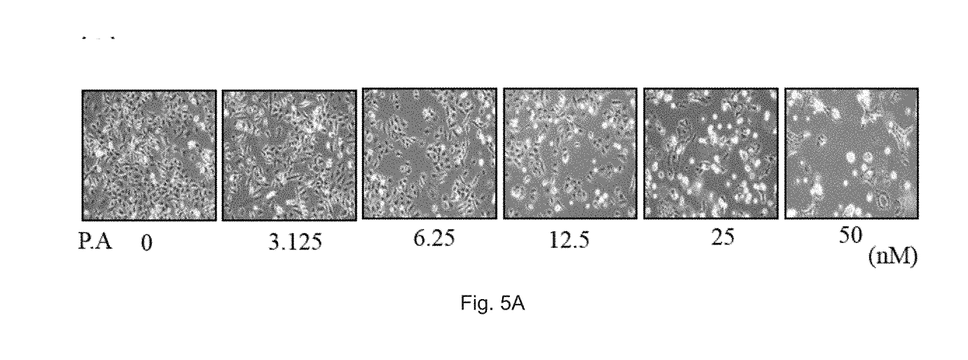

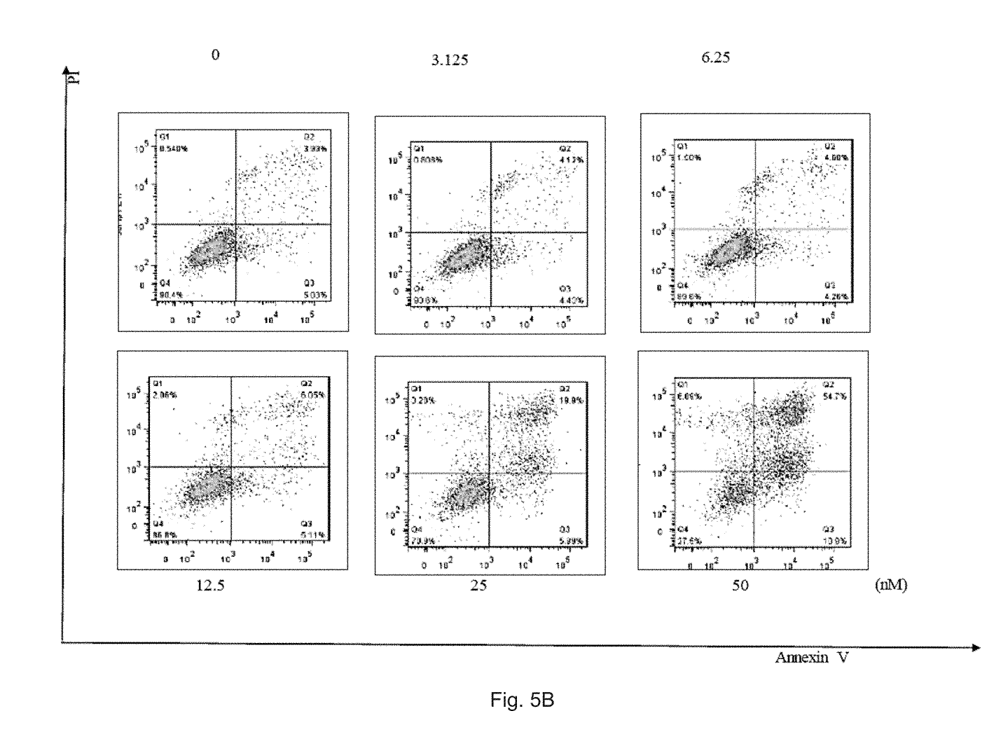

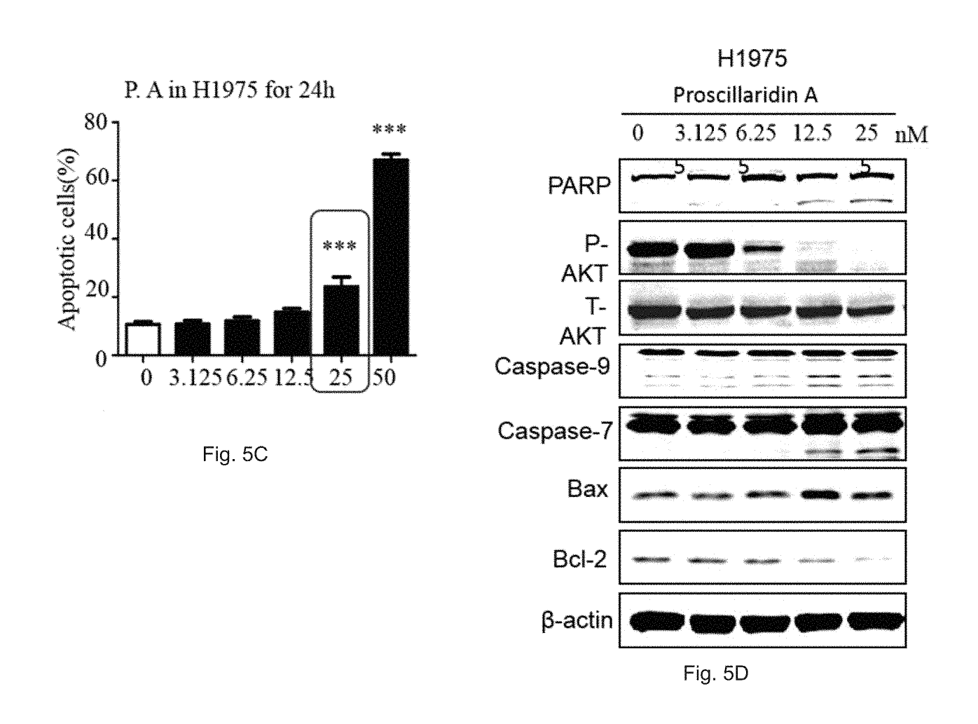

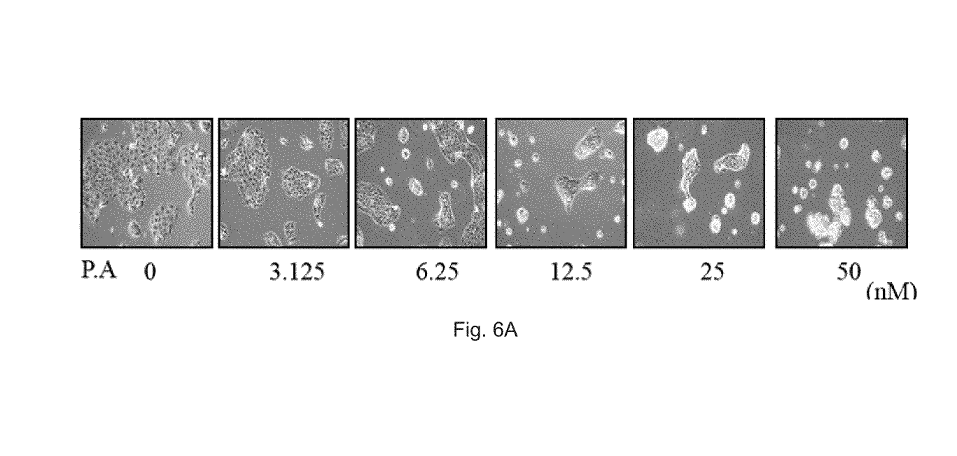

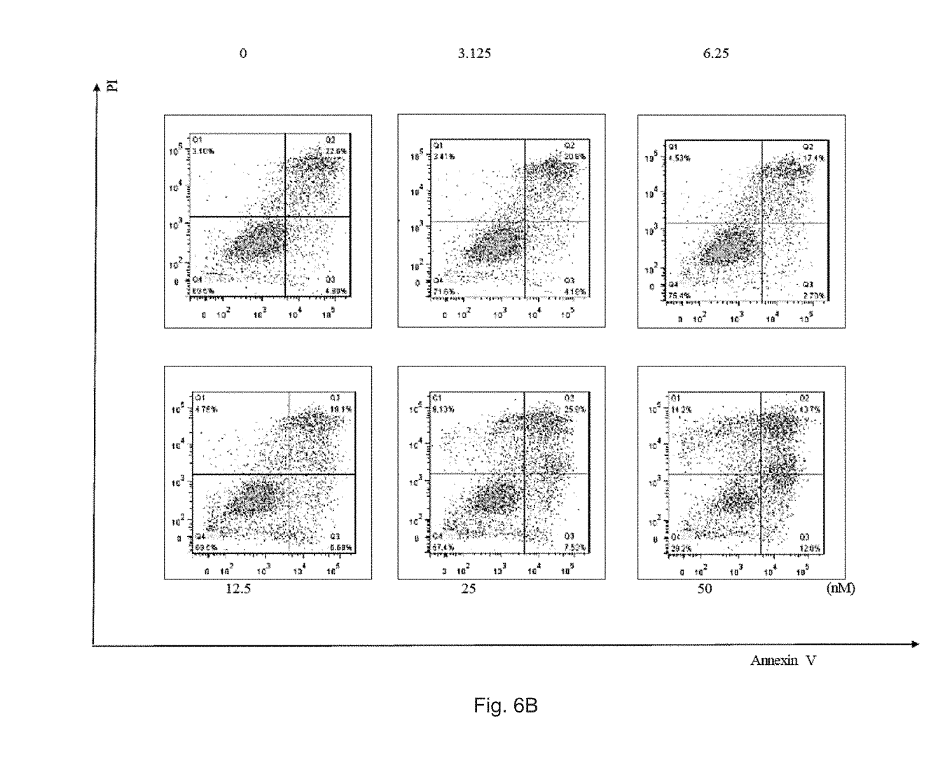

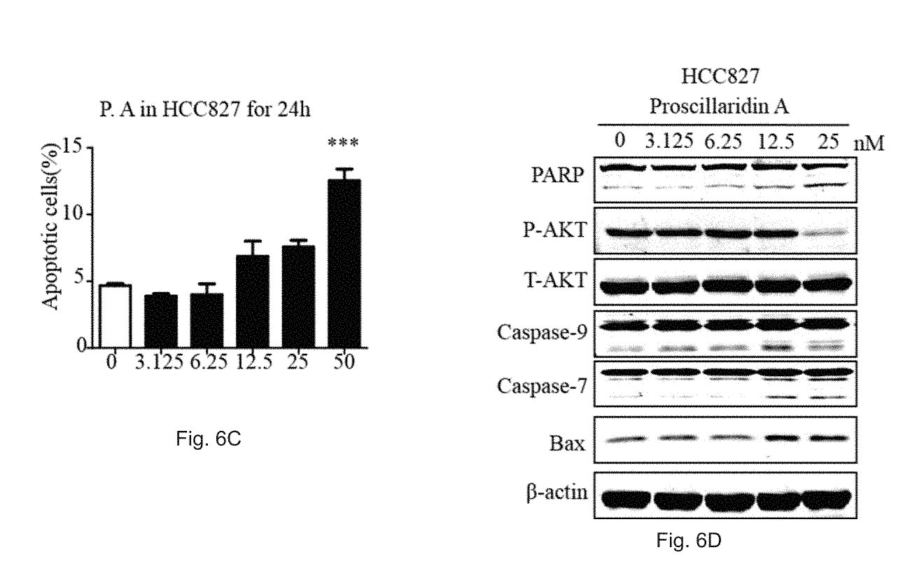

To determine whether Proscillaridin A can induce apoptosis in EGFR-dependent NSCLC cell lines, apoptosis in A549, H1975 and HCC827 after Proscillaridin A treatment has been measured. The apoptotic effect of Proscillaridin A has been determined by microscopic and flow cytometric analysis. As shown in FIG. 4 to FIG. 6, the results confirm that Proscillaridin A induces apoptosis in all three cell lines. For example, Proscillaridin A significantly induces apoptosis in A549 and H1975 starting at 12.5 nM and 25 nM separately increasing to about 70% apoptotic cells at 50 nM. In HCC827, Proscillaridin A induces apoptosis with an increase about 10% at 50 nM lower than the other two cell lines. As shown in FIG. 4 to FIG. 6, PARP, Caspase-7 and Caspase-9 were cleaved and activated. Pro-survival regulator AKT was inhibited and the pro-apoptotic proteins BAX was up-regulated while the anti-apoptotic proteins Bcl-2 was down-regulated.

After comparing the results, A549 and H1975 were chosen to do the following functional effect experiments of Proscillaridin A.

### Inhibition of Na⁺/K⁺ ATPase Activity and Increase of Ca²⁺ Levels

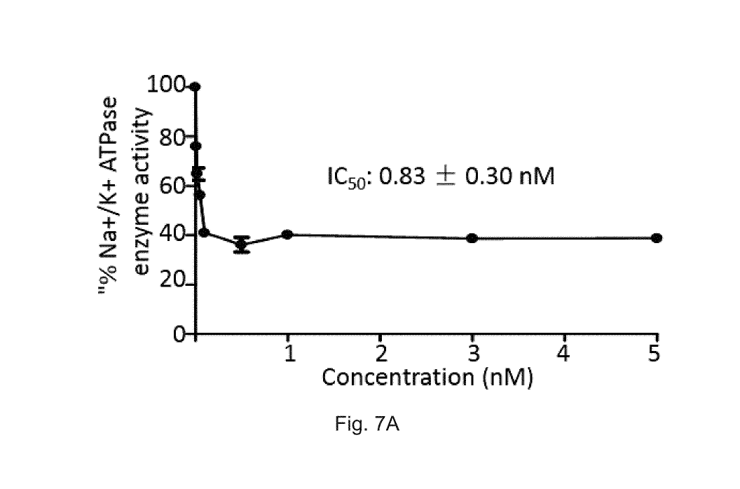

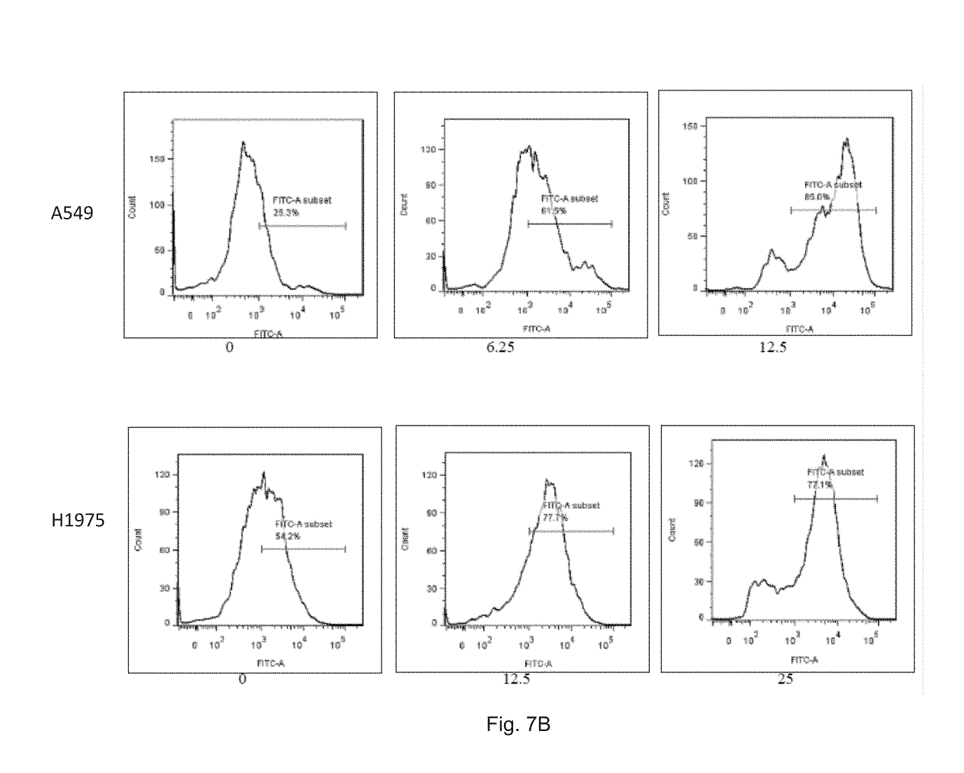

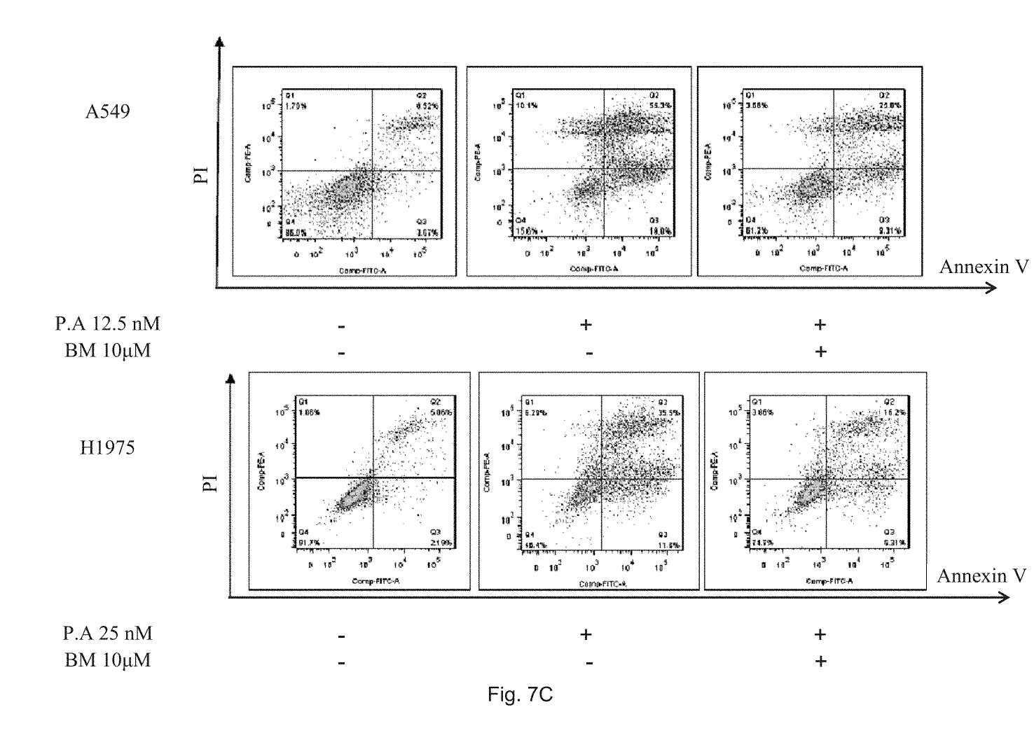

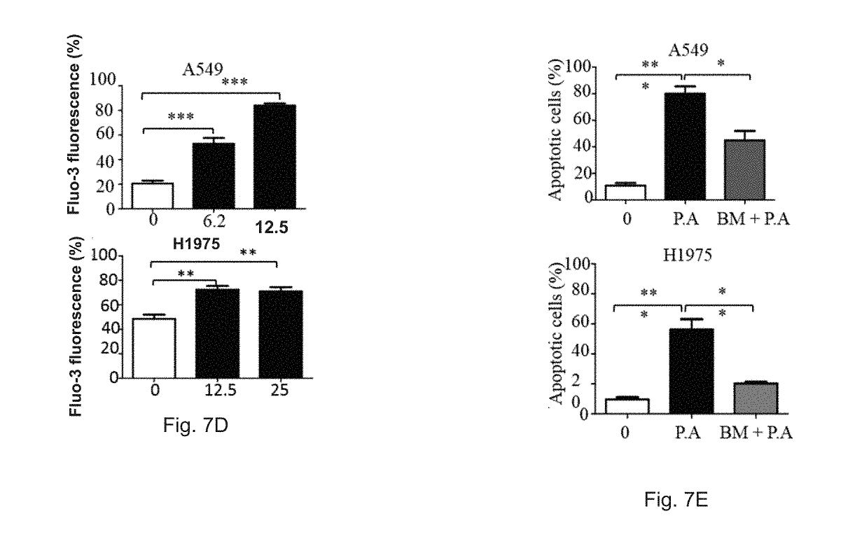

It has been determined whether Proscillaridin A can inhibit the Na⁺/K⁺ ATPase. Thus the enzyme activity was performed in an in vitro Na⁺/K⁺ ATPase assay. As shown in FIG. 7A, the IC₅₀ was 0.83±0.3 nM implying that Proscillaridin A can inhibit the Na⁺/K⁺ ATPase activity. As Proscillaridin A inhibited the Na⁺/K⁺ ATPase, the ionic level is assumed to change (Liu, J., Journal of Biological Chemistry 275 (2000) 27838-27844). Thus it has been evaluated whether the Na⁺/Ca²⁺ pump would be activated and the Ca²⁺ level would increase (Mcconkey, D. J., Cancer Research 60 (2000) 3807-3812). A549 and H1975 cells were stained with Fluo3-AM to monitor and the compare the cellular Ca²⁺ level. The results proved that Proscillaridin A remarkably increases the in vitro Ca²⁺ level upon Proscillaridin A treatment after 6 h (FIG. 7B).

To ascertain whether the Ca²⁺ levels elevation was important to Proscillaridin A induced apoptosis, the Ca²⁺ chelator BAPTA/AM (BM), which can decrease the in vitro Ca²⁺ level was applied to co-treat with Proscillaridin A. By flow cytometry, the results clearly show a significant decrease of apoptotic cells after the co-treatment of BM with Proscillaridin A (FIG. 7C). These results suggest that the elevation of Ca²⁺ level is an important mediator of Proscillaridin A induced apoptosis.

### Activation of AMPK Phosphorylation and JNK Phosphorylation and Down-Regulation of ACC Phosphorylation

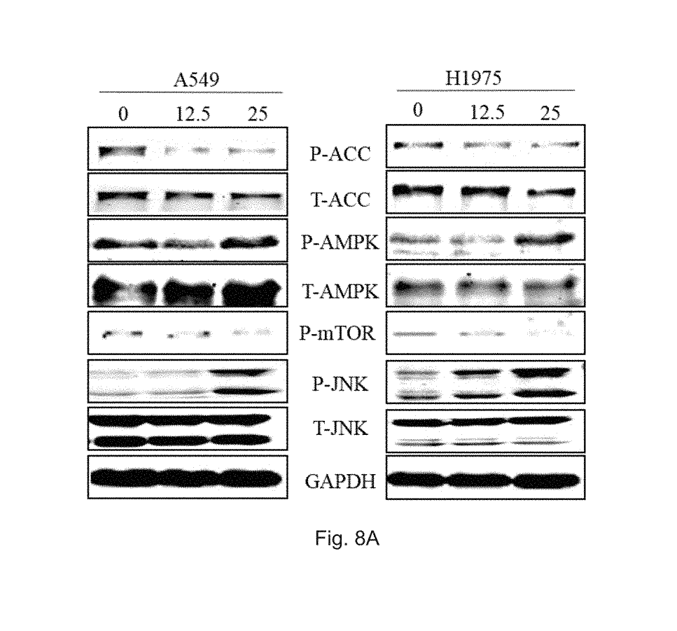

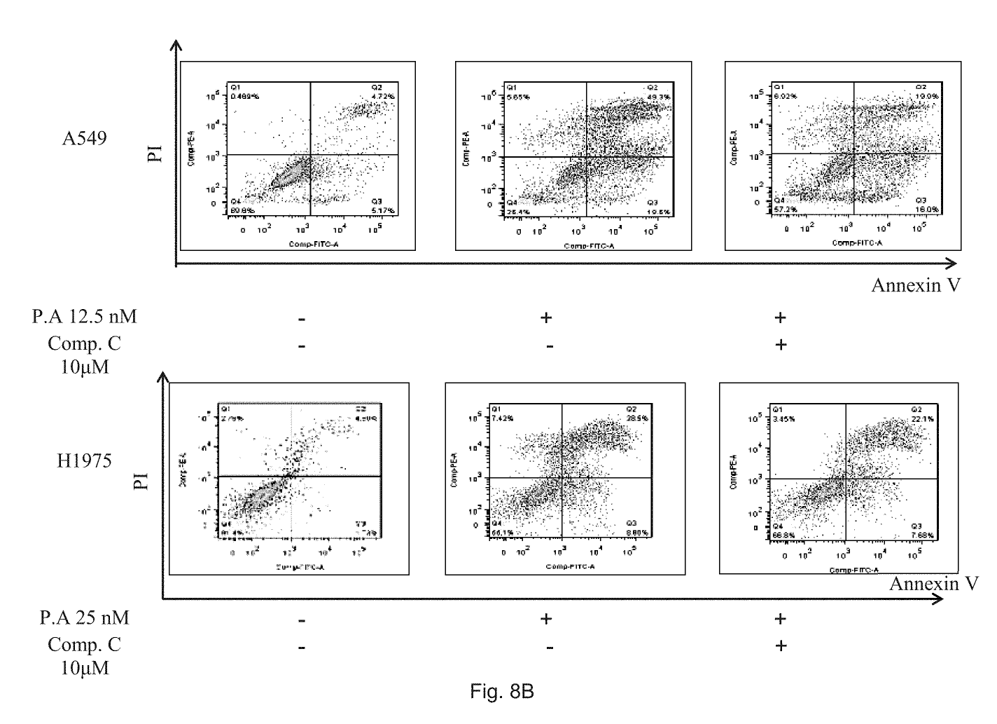

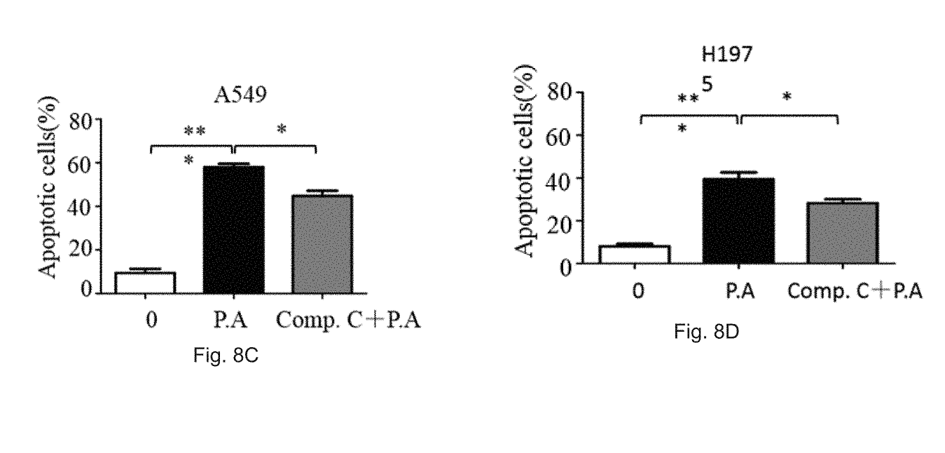

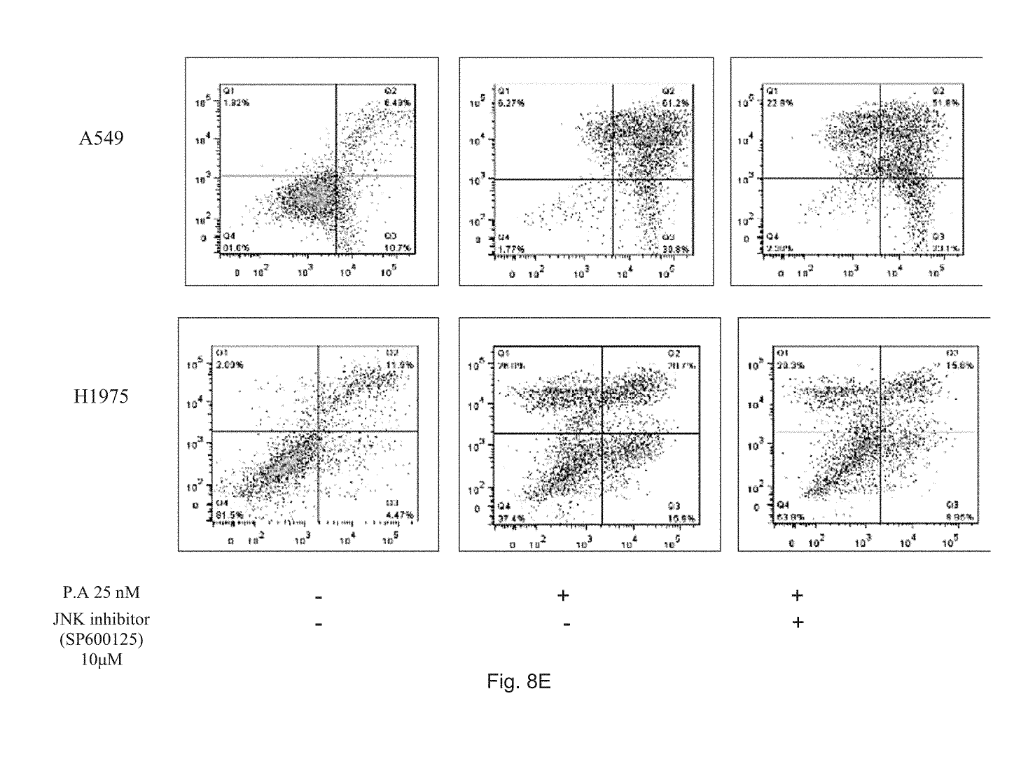

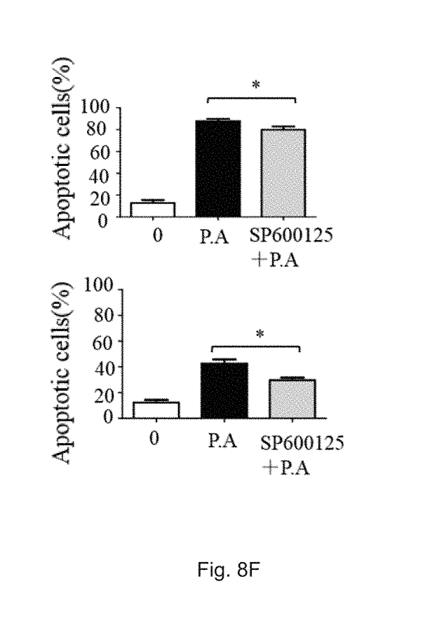

The induction of calcium influx is one of the important AMPK activation mechanism which requires the release of Ca²⁺ from the endoplasmic reticulum (ER). In FIG. 8A, Proscillaridin A significantly induced the phosphorylation of AMPK as well as its direst downstream target acetyl-CoA carboxylase (ACC). Then it has been evaluated whether the activation of AMPK was important to the Proscillaridin A-induced apoptosis. Compound C has been applied which is a specific inhibitor of AMPK to block the activation of AMPK and to examine the cell death level. The results suggest that the compound C partially weakened the apoptotic cells in A549 and H1975 cell lines (FIG. 8B). Thus, AMPK was also a key mediator of Proscillaridin A induced apoptosis.

It has been reported that AMPK is a major upstream regulator of mTOR, the activation of AMPK is able to suppress the activity of mTOR. As shown in FIG. 8A, Proscillaridin A inhibited the phosphorylation of mTOR. Since the c-Jun N-terminal kinase (JNK) is closely associated with ER stress and mTOR pathway and as the elevation of calcium level can induce ER stress, it has been evaluated whether the JNK was also an important mediator in the apoptosis mechanism of Proscillaridin A. Therefore, the activation of JNK with the treatment of Proscillaridin A has been examined. As shown in FIG. 8A, Proscillaridin A significantly activated the phosphorylation of JNK. To determine whether the activation of JNK was required for Proscillaridin A-induced apoptosis, JNK specific inhibitor SP600125 was used to inhibit the activation of JNK. Unlike the results above, the inhibition of JNK only slightly reduced the apoptotic cells after the co-treatment of SP600125 with Proscillaridin A.

### Up-Regulation of DR4 Expression and Inhibition of DR4′s Suppressor NF-κB Pathway

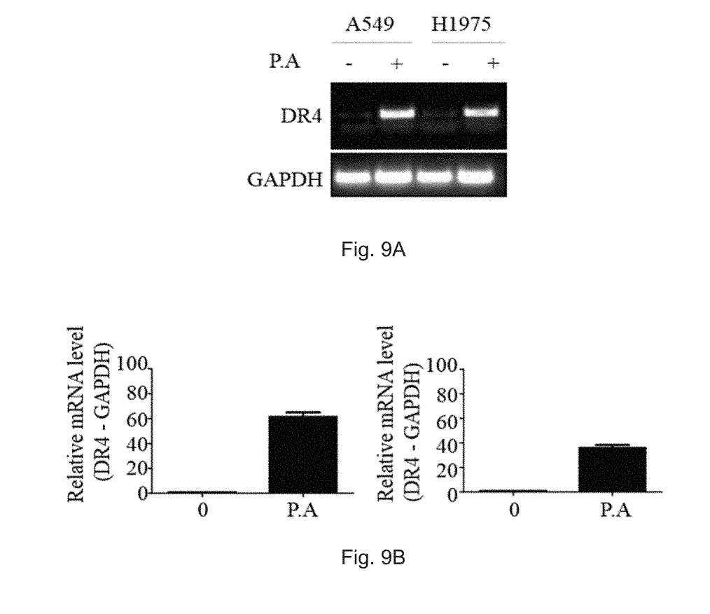

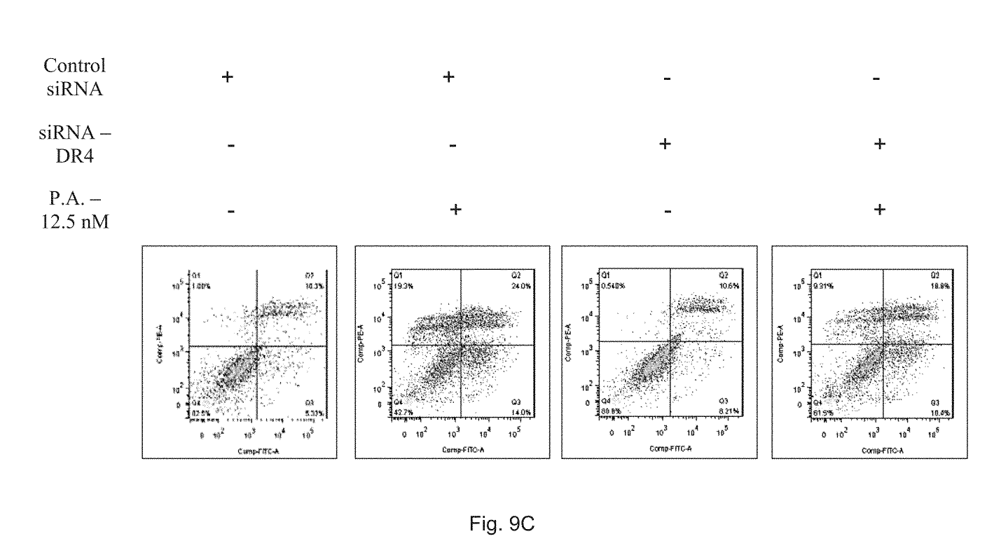

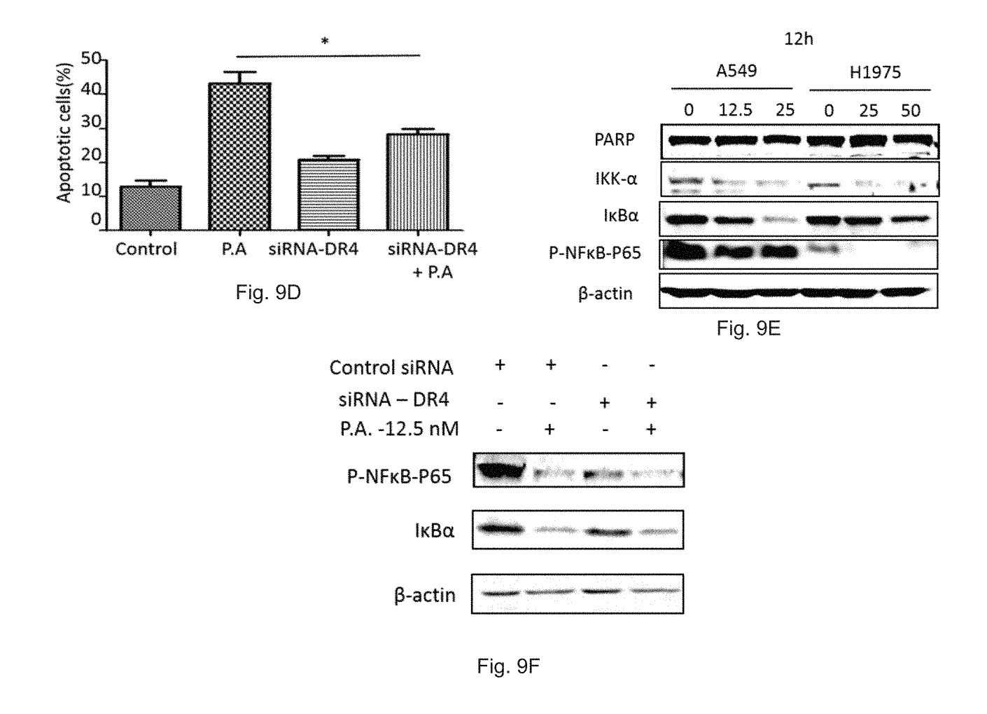

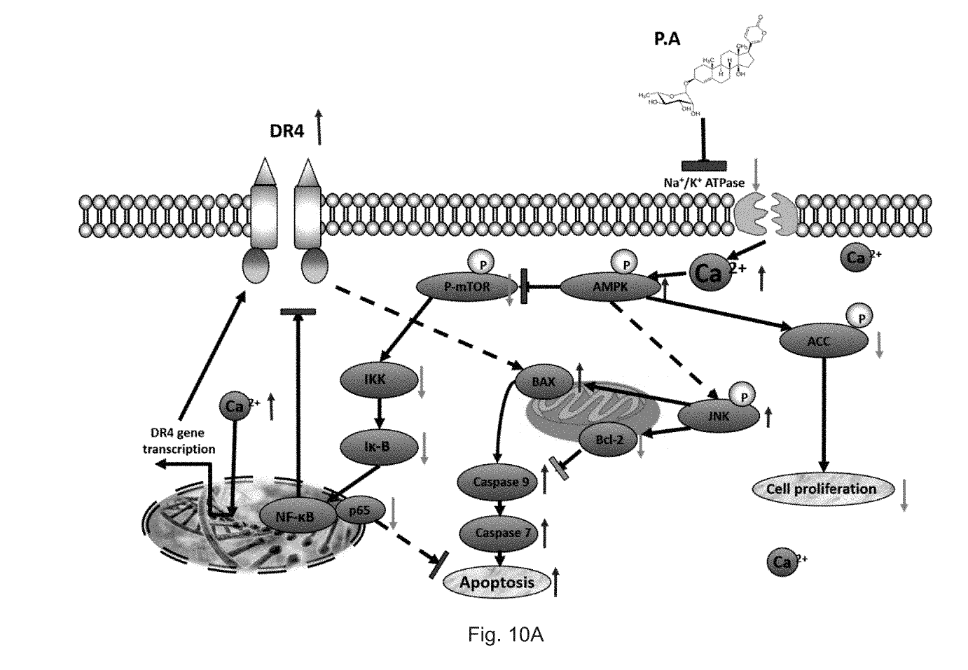

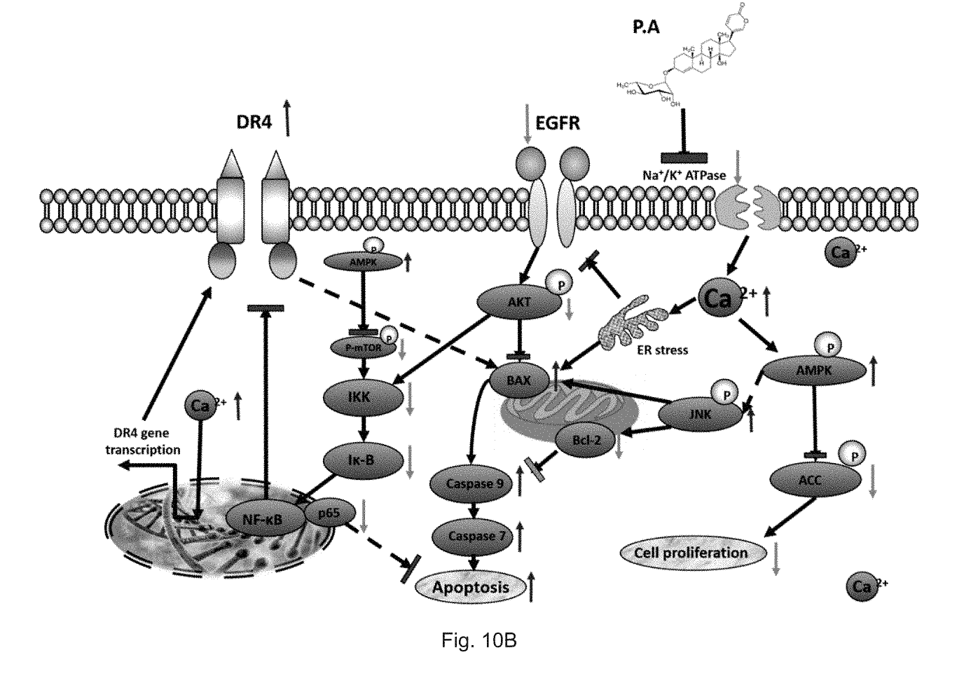

In one of the downstream of Ca²⁺ regulation pathways, Tumor Necrosis Factor (TNF) receptor is closely related to the apoptotic mechanism. Moreover, the TNF-related apoptosis inducing ligand (TRAIL) can only induce tumor cells to death not the normal cells via interacting with TRAIL-receptor1, also called as death receptor 4 (DR4). Moreover, up to date, there is no correlated study of effects of Proscillaridin A on DR4 pathway in NSCLC cells yet. Thus, the gene expression of DR4 after the treatment of Proscillaridin A has been investigated. As shown in FIG. 9A and B, both regular and quantitive RT-PCR results demonstrate the DR4′s expression was significantly elevated. In addition to this, when the DR4 was knocked down, the apoptotic cells following decreased (FIG. 9C and D). This demonstrates that DR4 is an important mediator of the apoptosis induced by Proscillaridin A.

At the same time, it has been evaluated whether after the treatment of Proscillaridin A the DR4's suppressor, NF-κB's activation, which at the same is reported the downstream of mTOR pathway is affected. The results reveal that the NF-κB pathway is significantly inhibited by Proscillaridin A in a dose-dependent manner (FIG. 9E).

Subsequently it has been determined whether the suppression of NF-κB pathway happened dependently or independently on the elevated expression of DR4. When the DR4 was knocked down, the decrease of NF-κB phosphorylation had no significant change (FIG. 9F). Therefore, this suggests that there is another way of down-regulation of NF-κB by Proscillaridin A.

1. A method of treating a subject suffering from non-small cell lung cancer comprising a step of administering an effective amount of a bufadienolide of Formula (I) to the subject: 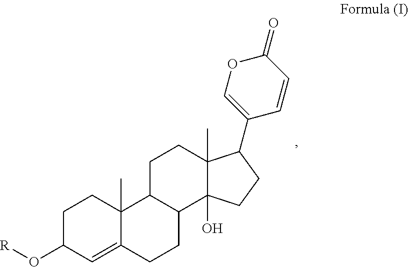
wherein R is selected from H and a glycoside moiety of 1 to 6 sugar residues; and
the cancer comprises cancer cells harboring an abnormality in the EGFR gene resulting from E746-A750del deletion in exon 19.

2. The method of claim 1, wherein the bufadienolide has the structure of Formula (II):

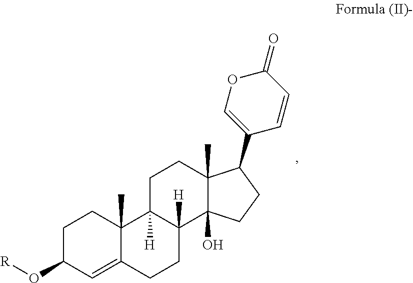

3. The method of claim 2, wherein R is a glycoside moiety of 1 to 3 sugar residues selected from the group consisting of L-rhamnose and D-glucose.

4. The method of claim 3, wherein the bufadienolide has the structure of Formula (III):

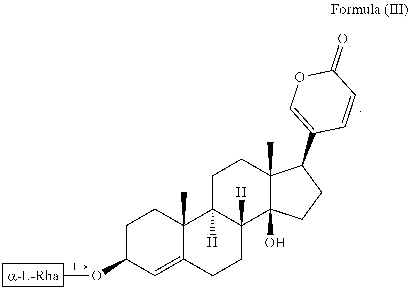

5. The method of claim 1, wherein the non-small cell lung cancer is an adenocarcinoma.

6. The method of claim 1, wherein the non-small cell lung cancer is Epidermal growth factor receptor (EGFR)-dependent.

7. The method of claim 1, wherein the non-small cell lung cancer cells further harbor an abnormality in the EGFR gene resulting from at least one of an exon 20 substitution and an exon 21 substitution.

8. The method of claim 7, wherein the abnormality in the EGFR gene further results from T790M substitution in exon 20.

9. The method of claim 1, wherein the bufadienolide is administered in form of a pharmaceutical composition comprising the bufadienolide and at least one pharmaceutically tolerable excipient selected from the group consisting of a diluent, a filler, a binder, a disintegrant, a lubricant, a coloring agent, a surfactant and a preservative.

10. A method of inhibiting the proliferation and inducing cell death of non-small cell lung cancer cells comprising a step of contacting said cells with an effective amount of a bufadienolide of Formula (I): 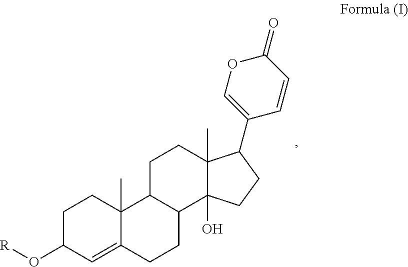
wherein R is selected from H and a glycoside moiety of 1 to 6 sugar residues; and
the cells harboring an abnormality in the EGFR gene result from E746-A750del deletion in exon 19.

11. The method of claim 10, wherein the bufadienolide has the structure of Formula (III):

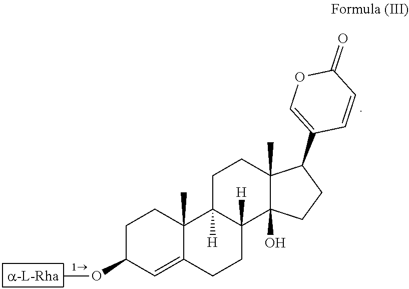

12. The method of claim 10, wherein the non-small cell lung cancer cells further harbor an abnormality in the EGFR gene resulting from at least one of an exon 20 substitution and an exon 21 substitution.

13. The method of claim 12, wherein the abnormality in the EGFR gene further results from T790M substitution in exon 20.

14. (canceled)

15. (canceled)

16. The method of claim 10, wherein the CC₅₀ value of the bufadienolide on non-cancerous lung cells is at least 10 times higher than the CC₅₀ on non-small cell lung cancer cells.

17. A method of inhibiting EGFR kinase activity in non-small cell lung cancer cells harboring an abnormality in the EGFR gene comprising a step of contacting said cells with an effective amount of a bufadienolide that comprises a structure of Formula (I): 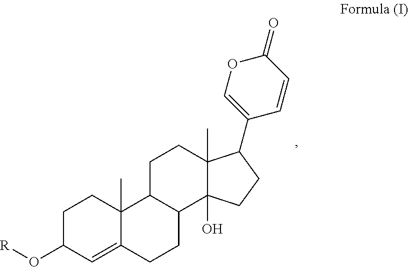
wherein R is selected from H and a glycoside moiety of 1 to 6 sugar residues; and
the abnormality in the EGFR gene results from E746-A750del deletion in exon 19.

18. The method of claim 17, wherein the bufadienolide has the structure of Formula (III):

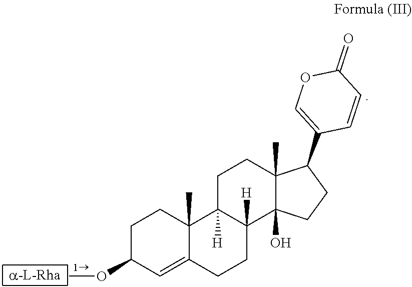

19. The method of claim 17, wherein the abnormality in the EGFR gene further results from at least one of an exon 20 substitution and an exon 21 substitution.

20. The method of claim 19, wherein the abnormality in the EGFR gene further results from T790M substitution in exon 20.
